(19) United States
(12) Patent Application Publication
(10) Pub. No.: US 2003/0017153 A1
(43) Pub. Date: Jan. 23, 2003

(54) Diagnostic indicator of thymic function

(76) Inventor: Richard Boyd, Prahran (AU)

Correspondence Address: IRELL & MANELLA LLP, 1800 AVENUE OF THE STARS, SUITE 900, LOS ANGELES, CA, 90067, US

(21) Appl. No.: 09/885,268

(22) Filed: Aug. 1, 2001

(30) Foreign Application Priority Data
Oct. 13, 2000 (AU) PRO745

(51) Int. Cl. A61K 38/09; A61K 39/395

(52) U.S. Cl. 424/145.1; 514/16; 514/17

(57) ABSTRACT

The present invention provides a method for determining whether a patient's immune system can be modified through stimulation of thymus function. In a preferred embodiment, sex steroids are ablated in the patient, and the resulting production of thymic factors is monitored. In particular, the level of these factors in the patient's blood stream is observed. In another embodiment, the level of new T cells is monitored. An early response, such as within hours or days of the ablation, indicates that the patient's thymus is disposed to regeneration through sex steroid ablation.

### FIELD OF THE INVENTION

The present invention is in the field of immunology. In particular, it relates to diagnosing the ability of a thymus to be reactivated by inhibition of the effects of sex steroids on the thymus.

### BACKGROUND OF THE INVENTION

The thymus is arguably the major organ in the immune system because it is the primary site of production of T lymphocytes. Its role is to attract appropriate bone marrow-derived precursor cells from the blood, and induce their commitment to the T cell lineage, including the gene rearrangements necessary for the production of the T cell receptor for antigen (TCR). Associated with this is a remarkable degree of cell division to expand the number of T cells and hence increase the likelihood that every foreign antigen will be recognized and eliminated. This enormous potential diversity means that for any single antigen the body might encounter, multiple lymphocytes will be able to recognize it with varying degrees of binding strength (affinity) and respond to varying degrees.

A strange feature of T cell recognition of antigen, however, is that, unlike B cells, the TCR only recognizes peptide fragments physically associated with MHC molecules. Normally this is self MHC (i.e., non-foreign MHC) and this ability is selected for in the thymus. This process is called positive selection and is an exclusive feature of cortical epithelial cells. If the TCR fails to bind to the self MHC/peptide complexes, the T cell dies by “neglect”—it needs some degree of signaling through the TCR for its continued maturation.

Following selection in the cortex, the developing thymocytes acquire functional maturation and migratory capacity and exit into the blood stream as naive (not yet having contacted antigen) T cells. They circulate between the lymph and blood in search of antigen. If after 3-4 weeks they haven't been stimulated, they become susceptible to deletion from the peripheral T cell pool by other recent thymic emigrants. This system of thymic export and peripheral T cell replacement provides a continual replenishment of the quality of T cells, with homeostasis maintaining the appropriate levels.

While the thymus is fundamental for a functional immune system, releasing ˜1% of its T cell content into the bloodstream per day, one of the apparent anomalies of mammals is that this organ undergoes severe atrophy as a result of sex steroid production. This can begin even in young children but is profound from the time of puberty. For normal healthy individuals this loss of production and release of new T cells does not always have immediate clinical consequences. In fact although the aged thymus is atrophic and consists of less than 1% of its young counterpart, it still continues to release a very low level of new T cells into the blood stream.

These are insufficient, however, to maintain the optimal levels of peripheral T cell subsets. But this does mean that the thymus is not completely dormant, raising the possibility that it could be the target of therapy. With progressive aging, the decline in thymic export means that the status of peripheral T cells undergoes progressive change both quantitatively and qualitatively. On the one hand there is a gradual decrease in absolute T cell numbers in the blood with age as they die off through lack of stimulation. On the other hand, with each antigen contact, the relevant antigen-specific naive T cells (those which have not yet encountered antigen) are stimulated and proliferate. A subset will progress to be effector cells and rid the body of the pathogen, but these eventually die through antigen-induced cell death. Another subset will convert to memory cells and provide long term protection against future contacts with that pathogen. Hence, there is a decrease in the levels of naive T cells and thus a reduced ability to respond to antigen.

Aging also results in a selective decline in Th cells (characterized by expression of CD4) relative to Tc cells (expressing CD8) and imbalances in the ratios of Th1 to Th2 cells. This does not occur in the normal young because, as mentioned above, there is a continual supply of new T cells being exported from the thymus, which in turn provides a continual replenishment of the naive T cell pool in the periphery.

Aging is not the only condition which results in T cell loss—this also occurs very severely for example in HIV/AIDS and following chemotherapy or radiotherapy. Again, in the young with an active thymus, the recovery of the immune system (through recovery of T cell mediated immunity) occurs relatively quickly (2-3 months), compared to post-puberty, when it can take 1-2 years because of the atrophic thymus.

There are thus several parameters which can influence the nature and extent of immune responses: the level and type of antigen, the site of vaccination, the availability of appropriate APC (antigen presenting cells), the general health of the individual and the status of the T and B cell pools. Of these, T cells are the most vulnerable because of the marked sex steroid induced shut-down in thymic export which becomes profound from the onset of puberty.

Any vaccination program should therefore only be logically undertaken when the level of potential responder T cells is optimal in terms of both the level of naive T cells representing a broad repertoire of specificity and the correct ratios of Th1 to Th2 cells and Th to Tc cells. The level and type of cytokines should also be manipulated to be appropriate for the desired response.

The ability to reactivate the atrophic thymus through inhibition of LHRH signaling to the pituitary provides a potent means of generating a new cohort of naive T cells with a diverse repertoire of TCR types. This process effectively reverts the thymus to its prepubertal state, and does so by using the normal regulatory molecules and pathways which lead to optimal thymopoiesis.

The thymus is influenced to a great extent by its bi-directional communication with the neuroendocrine system (Kendall, 1988). Of particular importance is the interplay between the pituitary, adrenals and gonads on thymic function including both trophic (TSH and GH) and atrophic effects (LH, FSH and ACTH) (Kendall, 1988; Homo-Delarche, 1991). Indeed one of the characteristic features of thymic physiology is the progressive decline in structure and function which is commensurate with the increase in circulating sex steroid production around puberty (Hirokawa and Makinodan, 1975; Tosi et al, 1982 and Hirokawa, et al., 1994). The precise target of the hormones and the mechanism by which they induce thymus atrophy is yet to be determined.

Since the thymus is the primary site for the production and maintenance of the peripheral T cell pool, this atrophy has been widely postulated as the primary cause of an increased incidence of immune-based disorders in the elderly. In particular, deficiencies of the immune system illustrated by a decrease in T-cell dependent immune functions such as cytolytic T-cell activity and mitogenic responses, are reflected by an increased incidence of immunodeficiency, autoimmunity and tumor load in later life (Hirokawa, 1998).

The impact of thymus atrophy is reflected in the periphery, with reduced thymic input to the T cell pool resulting in a less diverse T cell receptor (TCR) repertoire. Altered cytokine profile (Hobbs et al., 1993; Kurashima et al., 1995), changes in CD4⁺ and CD8⁺ subsets, and a bias towards memory as opposed to naïve T cells (Mackall et al., 1995) are also observed. Furthermore, the efficiency of thymopoiesis is impaired with age such that the ability of the immune system to regenerate normal T-cell numbers after T-cell depletion is eventually lost (Mackall et al., 1995).

However, recent work by Douek et al. (1998) has shown presumably thymic output to occur even in old age in humans. Excisional DNA products of TCR gene-rearrangement were used to demonstrate circulating, de novo produced naive T cells after HIV infection in older patients. The rate of this output and subsequent peripheral T cell pool regeneration needs to be further addressed since patients who have undergone chemotherapy show a greatly reduced rate of regeneration of the T cell pool, particularly CD4⁺T cells, in post-pubertal patients compared to those who were pre-pubertal (Mackall et al, 1995). This is further exemplified in recent work by Timm and Thoman (1999), who have shown that although CD4⁺ T cells are regenerated in old mice post BMT (bone marrow transplant), they appear to show a bias towards memory cells due to the aged peripheral microenvironment, coupled to poor thymic production of naive T cells.

The thymus essentially consists of developing thymocytes interspersed within the diverse stromal cells (predominantly epithelial cell subsets) which constitute the microenvironment and provide the growth factors and cellular interactions necessary for the optimal development of the T cells. The symbiotic developmental relationship between thymocytes and the epithelial subsets that controls their differentiation and maturation (Boyd et al., 1993) means sex-steroid inhibition could occur at the level of either cell type which would then influence the status of the other. It is less likely that there is an inherent defect within the thymocytes themselves since previous studies, utilizing radiation chimeras, have shown that bone marrow (BM) stem cells are not affected by age (Hirokawa, 1998; Mackall and Gress, 1997) and have a similar degree of thymus repopulation potential as young BM cells. Furthermore, thymocytes in aged animals retain their ability to differentiate to at least some degree (Mackall and Gress, 1997; George and Ritter, 1996; Hirokawa et al., 1994). However, recent work by Aspinall (1997), has shown a defect within the precursor CD3⁻CD4⁻CD8⁻ triple negative (TN) population occurring at the stage of TCRγ chain gene-rearrangement.

Skewing of Developing TCR Repertoire Towards, or Away from, Specific Antigens.

The ability to enhance the uptake into the thymus of haematopoietic precursor cells means that the nature and type of dendritic cells can be manipulated. For example the precursors can be transfected with specific gene(s) which eventually become expressed in the dendritic cells in the thymus (and elsewhere in the body). Such genes can include those which encode specific antigens for which an immune response would be detrimental, e.g., autoimmune diseases, allergies and graft antigens

The genes can also encode antigens (also as peptides) for which an immune response is desired, e.g., tumor cells and invading microorganisms. In the latter case the level and affinity of the peptide would be manipulated to be low enough so as not to induce negative selection, but high enough to promote positive selection. We have shown that positive selection can involve multiple cell types: the cortical epithelium provides the specific differentiation molecules, and third party cells the MHC/peptide ligands.

The precursors can also be genetically modified by adding or deleting genes, such as those coding for soluble regulatory molecules, such as chemokines, cytokines and other molecules affecting any aspect of thymopoiesis and T cell development, activation, positive or negative selection, migration, and general status. This approach can be used to promote or retard thymic development or T cell responsiveness. It can be used to skew the T cell repertoire to specific antigens to create, for example, anti-viral and anti-tumor defenses. This approach can also be used to modulate the nature, organization and function of the thymic microenvironment.

Induction of Tolerance

The most effective means of generating tolerance to self is through intra-thymic deletion (or anergy or induction of negative regulatory cells) of the potentially self reactive cells through negative selection, mediated most efficiently by intrathymic dendritic cells. As a corollary, the establishment of tolerance to exogenous or nominal antigens could be best achieved if dendritic cells expressing this antigen could be incorporated into the thymus. This form of tolerance may also be made more effective through the advent of inhibitory immunoregulatory cells. The mechanisms underlying the development of the latter, however, are poorly understood, but again occur in the thymus and could involve dendritic cells.

In the case of hyperreactive T cells for which the target antigen is known, the haematopoietic stem cells can be transfected with the gene encoding the specific antigen. When these cells develop into dendritic cells in the thymus they will delete any new T cells arising which are potentially reactive to the nominal antigen.

The enormous clinical benefits to be gained through restoration of thymic function, represent an important strategy for the treatment of immunodeficiencies, particularly in the elderly, HIV patients and patients following chemotherapy. Furthermore patients who have functionally abnormal T cells can now be treated to remove all T cells, thereby stopping the disease, and then have their normal immunity restored by reactivation of thymic function by inhibition of sex steroid production. In the case of vaccination programs, the reactivation of the thymus will have profound improvements on the status of T cells and hence the nature, extent and quality of immune responses. Additionally, through presentation of donor cells during reactivation of the thymus, T cell populations can be modified to allow for tolerance of allogeneic and xenogeneic grafts. Moreover, regenerating populations of T cells can be genetically modified through gene therapy during thymic reactivation.

### SUMMARY OF THE INVENTION

The present invention provides a diagnostic method for determining the susceptibility of a thymus to regeneration by inhibition of sex steroid production. In a preferred embodiment, the method provides an early determination of this susceptibility, preferably within a week, more preferably within 4 to 5 days, even more preferably within 2-3 days, and most preferably with 24 hours of initiation of inhibition.

In a preferred embodiment, inhibition is caused by administering an LHRH agonist. Preferably a quick-acting antagonist such as Abarelix or Cetrorelix is administered. In an alternative embodiment, inhibition is caused by administering an LHRH agonist such as Zoladex or Leupron.

In one embodiment, the diagnosis is accomplished by measuring the amount of thymic induced factors in a blood sample of the patient before and after initiation of inhibition.

In yet another embodiment, the invention is used to identify previously unidentified thymic factors.

In another embodiment, the diagnosis is accomplished by measuring thymic activity. In addition to the above, this will be achieved by determining levels of newly produced T cells identified by the presence in these cells of small circles of DNA termed T cell receptor excision circles (TREC's). These TREC's are produced as a normal part of T cell development in the thymus, in particular as a result of gene rearrangements in the formation of the T cell receptor for antigen. Basic increases in total T cell number (as measured by flow cytometry staining for CD3, CD4 and CD8) and shifts in their in vitro responsiveness to stimulation with anti-CD3 cross-linking can also be used to monitor thymic function but they are expected to take several days to weeks before any changes may be detectable.

### FIGURES

FIG. 1: Changes in thymocyte number pre- and post-castration. Thymus atrophy results in a significant decrease in thymocyte numbers with age. By 2 weeks post-castration, cell numbers have increased to young adult levels. By 3 weeks post-castration, numbers have significantly increased from the young adult and they are stabilized by 4 weeks post-castration. ***=Significantly different from young adult (2 month) thymus, p<0.001

FIG. 2: (A) Spleen numbers remain constant with age and post-castration. The B:T cell ratio in the periphery also remains constant (B), however, the CD4:CD8 ratio decreases significantly (p<0.00l) with age and is restored to normal young levels by 4 weeks post-castration.

FIG. 3: Fluorescence Activated Cell Sorter (FACS) profiles of CD4 vs. CD8 thymocyte populations with age and post-castration. Percentages for each quadrant are given above each plot. Subpopulations of thymocytes remain constant with age and there is a synchronous expansion of thymocytes following castration.

FIG. 4A: Proliferation of thymocytes as detected by incorporation of a pulse of BrdU. Proportion of proliferating thymocytes remains constant with age and following castration.

FIG. 4B: Effects of age and castration on proliferation of thymocyte subsets. (A) Proportion of each subset that constitutes the total proliferating population—The proportion of CD8+ T cells within the proliferating population is significantly increased. (B) Percentage of each subpopulation that is proliferating—The TN and CD8 Subsets have significantly less proliferation at 2 years than at 2 months. At 2 weeks post-castration, the TN population has returned to normal young levels of proliferation while the CD8 population shows a significant increase in proliferation. The level is equivalent to the normal young by 4 weeks post-castration. (C) Overall TN proliferation remains constant with age and post-castration, however, the significant decrease in proliferation of the TN1 subpopulation with age, is not returned to normal levels by 4 weeks post-castration (D). ***=Highly significant, p<0.001, **=significant, p<0.01

FIG. 5: Mice were injected intrathymically with FITC. The number of FITC+ cells in the periphery were calculated 24 hours later. Although the proportion of recent thymic migrants (RTE) remained consistently about 1% of thymus cell number age but was significantly reduced at 2 weeks post-castration, there was a significant (p<0.01) decrease in the RTE cell numbers with age. Following castration, these values were increasing although still significantly lower than young mice at 2 weeks post-castration. With age, a significant increase in the ratio of CD4+ to CD8+ RTE was seen and this was normalized by 1 week post-castration.

FIG. 6: Changes in thymus, spleen and lymph node cell numbers following treatment with cyclophosphamide, a chemotherapy agent. Note the rapid expansion of the thymus in castrated animals when compared to the non-castrate (cyclophosphamide alone) group at 1 and 2 weeks post-treatment. In addition, spleen and lymph node numbers of the castrate group were well increased compared to the cyclophosphamide alone group. By 4 weeks, cell numbers are normalized. (n=3-4 per treatment group and time point).

FIG. 7: Changes in thymus, spleen and lymph node cell numbers following irradiation (625 Rads) one week after surgical castration. Note the rapid expansion of the thymus in castrated animals when compared to the non-castrate (irradiation alone) group at 1 and 2 weeks post-treatment. (n=3-4 per treatment group and time point).

FIG. 8: Changes in thymus, spleen and lymph node cell numbers following irradiation and castration on the same day. Note the rapid expansion of the thymus in castrated animals when compared to the non-castrate group at 2 weeks post-treatment. However, the difference observed is not as obvious as when mice were castrated 1 week prior to treatment (FIG. 7). (n=3-4 per treatment group and time point).

FIG. 9: Changes in thymus, spleen and lymph node cell numbers following treatment with cyclophosphamide, a chemotherapy agent, and surgical or chemical castration performed on the same day. Note the rapid expansion of the thymus in castrated animals when compared to the non-castrate (cyclophosphamide alone) group at 1 and 2 weeks post-treatment. In addition, spleen and lymph node numbers of the castrate group were well increased compared to the cyclophosphamide alone group. (n=3-4 per treatment group and time point). Chemical castration is comparable to surgical castration in regeneration of the immune system post-cyclophosphamide treatment.

FIG. 10: Lymph node cellularity following foot-pad immunization with Herpes Simplex Virus-1 (HSV-1). Note the increased cellularity in the aged post-castration as compared to the aged non-castrated group. Bottom graph illustrates the overall activated cell number as gated on CD25 vs. CD8 cells by FACS.

FIG. 11: Representative examples of flow cytometry dot plots illustrating activated cell proportions in lymph nodes following Herpes Simplex Virus-1 (HSV-1) infection. Activated cells are CD25+CD8+ “Non-immune” are pooled popliteal lymph node cells from control uninfected young (2 months); “old immune” are pooled popliteal lymph node cells from uninfected old (˜18 months old) mice. “Young immune” is a representative example a popliteal lymph node from a young mice infected 5 days earlier with HSV-1 in the lower hind leg.

FIG. 12A: Vβ10 expression on CTL in activated LN following HSV-1 inoculation. Note the diminution of a clonal response in aged mice and the reinstatement of the expected response post-castration.

FIG. 12B: Popliteal lymph nodes were removed from mice immunized with HSV-1 and cultured for 3 days. CTL assays were performed with non-immunized mice as control for background levels of lysis (as determined by ⁵¹Cr-release). Results are expressed as mean of 8 mice, in triplicate ±1 SD. Aged mice showed a significant (p≦0.01, *) reduction in CTL activity at an E:T ratio of both 10:1 and 3:1 indicating a reduction in the percentage of specific CTL present within the lymph nodes. Castration of aged mice restored the CTL response to young adult levels.

FIG. 13: Changes in thymus, spleen, lymph node and bone marrow cell numbers following bone marrow transplantation of Ly5 congenic mice. Note the rapid expansion of the thymus in castrated animals when compared to the non-castrate group at all time points post-treatment. In addition, spleen and lymph node numbers of the castrate group were well increased compared to the cyclophosphamide alone group. (n=3-4 per treatment group and time point). Castrated mice had significantly increased congenic (Ly5.2) cells compared to non-castrated animals (data not shown).

FIG. 14: Changes in thymus cell number in castrated and noncastrated mice after fetal liver reconstitution. (n=3-4 for each test group.) (A) At two weeks, thymus cell number of castrated mice was at normal levels and significantly higher than that of noncastrated mice (*p≦0.05). Hypertrophy was observed in thymuses of castrated mice after four weeks. Noncastrated cell numbers remain below control levels. (B) CD45.2⁺ cells—CD45.2⁺ is a marker showing donor derivation. Two weeks after reconstitution donor-derived cells were present in both castrated and noncastrated mice. Four weeks after treatment approximately 85% of cells in the castrated thymus were donor-derived. There were no donor-derived cells in the noncastrated thymus.

FIG. 15: FACS profiles of CD4 versus CD8 donor derived thymocyte populations after lethal irradiation and fetal liver reconstitution, followed by surgical castration. Percentages for each quadrant are given to the right of each plot. The age matched control profile is of an eight month old Ly5.1 congenic mouse thymus. Those of castrated and noncastrated mice are gated on CD45.2⁺ cells, showing only donor derived cells. Two weeks after reconstitution subpopulations of thymocytes do not differ between castrated and noncastrated mice.

FIG. 16: Myeloid and lymphoid dendritic cell (DC) number after lethal irradiation, fetal liver reconstitution and castration. (n=3-4 mice for each test group.) Control (white) bars on the following graphs are based on the normal number of dendritic cells found in untreated age matched mice. (A) Donor-derived myeloid dendritic cells—Two weeks after reconstitution DC were present at normal levels in noncastrated mice. There were significantly more DC in castrated mice at the same time point. (*p≦0.05). At four weeks DC number remained above control levels in castrated mice. (B) Donor-derived lymphoid dendritic cells—Two weeks after reconstitution DC numbers in castrated mice were double those of noncastrated mice. Four weeks after treatment DC numbers remained above control levels.

FIG. 17: Changes in total and CD45.2⁺ bone marrow cell numbers in castrated and noncastrated mice after fetal liver reconstitution. (n=3-4 mice for each test group.) (A) Total cell number—Two weeks after reconstitution bone marrow cell numbers had normalized and there was no significant difference in cell number between castrated and noncastrated mice. Four weeks after reconstitution there was a significant difference in cell number between castrated and noncastrated mice (*p≦0.05). (B) CD45.2⁺ cell number. There was no significant difference between castrated and noncastrated mice with respect to CD45.2⁺ cell number in the bone marrow two weeks after reconstitution. CD45.2⁺ cell number remained high in castrated mice at four weeks. There were no donor-derived cells in the noncastrated mice at the same time point.

FIG. 18: Changes in T cells and myeloid and lymphoid derived dendritic cells (DC) in bone marrow of castrated and noncastrated mice after fetal liver reconstitution. (n=3-4 mice for each test group.) Controls (white) bars on the following graphs are based on the normal number of T cells and dendritic cells found in untreated age matched mice. (A) T cell number—Numbers were reduced two and four weeks after reconstitution in both castrated and noncastrated mice. (B) Donor derived myeloid dendritic cells—Two weeks after reconstitution DC cell numbers were normal in both castrated and noncastrated mice. At this time point there was no significant difference between numbers in castrated and noncastrated mice. (C) Donor-derived lymphoid dendritic cells—Numbers were at normal levels two and four weeks after reconstitution. At two weeks there was no significant difference between numbers in castrated and noncastrated mice.

FIG. 19: Change in total and donor (CD45.2⁺) spleen cell numbers in castrated and noncastrated mice after fetal liver reconstitution. (n=3-4 mice for each test group.) (A) Total cell number—Two weeks after reconstitution cell numbers were decreased and there was no significant difference in cell number between castrated and noncastrated mice. Four weeks after reconstitution cell numbers were approaching normal levels in castrated mice. (B) CD45.2⁺ cell number—There was no significant difference between castrated and noncastrated mice with respect to CD45.2⁺ cell number in the spleen, two weeks after reconstitution. CD45.2⁺ cell number remained high in castrated mice at four weeks. There were no donor-derived cells in the noncastrated mice at the same time point.

FIG. 20: Splenic T cells and myeloid and lymphoid derived dendritic cells (DC) in castrated and non-castrated mice after fetal liver reconstitution. (n=3-4 mice for each test group.) Control (white) bars on the following graphs are based on the normal number of T cells and dendritic cells found in untreated age matched mice. (A) T cell number—Numbers were reduced two and four weeks after reconstitution in both castrated and noncastrated mice. (B) Donor derived (CD45.2⁺) myeloid dendritic cells—two and four weeks after reconstitution DC numbers were normal in both castrated and noncastrated mice. At two weeks there was no significant difference between numbers in castrated and noncastrated mice. (C) Donor-derived (CD45.2⁺) lymphoid dendritic cells-numbers were at normal levels two and four weeks after reconstitution. At two weeks there was no significant difference between numbers in castrated and noncastrated mice.

FIG. 21: Changes in total and donor (CD45.2⁺) lymph node cell numbers in castrated and noncastrated mice after fetal liver reconstitution. (n=3-4 for each test group.) (A) Total cell numbers—Two weeks after reconstitution cell numbers were at normal levels and there was no significant difference between castrated and noncastrated mice. Four weeks after reconstitution cell numbers in castrated mice were at normal levels. (B) CD45.2⁺ cell number—There was no significant difference between castrated and noncastrated mice with respect to donor CD45.2⁺ cell number in the lymph node two weeks after reconstitution. CD45.2 cell number remained high in castrated mice at four weeks. There were no donor-derived cells in the noncastrated mice at the same point.

FIG. 22: Changes in T cells and myeloid and lymphoid derived dendritic cells (DC) in the mesenteric lymph nodes of castrated and non-castrated mice after fetal liver reconstitution. (n=3-4 mice for each test group.) Control (white) bars are the number of T cells and dendritic cells found in untreated age matched mice. (A) T cell number—Numbers were reduced two and four weeks after reconstitution in both castrated and noncastrated mice. (B) Donor derived myeloid dendritic cells were normal in both castrated and noncastrated mice. At four weeks they were decreased. At two weeks there was no significant difference between numbers in castrated and noncastrated mice. (C) Donor-derived lymphoid dendritic cells—Numbers were at normal levels two and four weeks after reconstitution. At two weeks there was no significant difference between numbers in castrated and noncastrated mice.

FIG. 23: The phenotypic composition of peripheral blood lymphocytes was analyzed in patients (all >60 years) undergoing LHRH agonist treatment for prostate cancer. Patient samples were analyzed before treatment and 4 months after beginning LHRH agonist treatment. Total lymphocyte cell numbers per ml of blood were at the lower end of control values before treatment in all patients. Following treatment, 6/9 patients showed substantial increases in total lymphocyte counts (in some cases a doubling of total cells was observed). Correlating with this was an increase in total T cell numbers in 6/9 patients. Within the CD4⁺ subset, this increase was even more pronounced with 8/9 patients demonstrating increased levels of CD4 T cells. A less distinctive trend was seen within the CD8⁺ subset with 4/9 patients showing increased levels; albeit generally to a smaller extent than CD4⁺ T cells.

FIG. 24: Analysis of patient blood before and after LHRH-agonist treatment demonstrated no substantial changes in the overall proportion of T cells, CD4 or CD8 T cells, and a variable change in the CD4:CD8 ratio following treatment. This indicates the minimal effect of treatment on the homeostatic maintenance of T cell subsets despite the substantial increase in overall T cell numbers following treatment. All values were comparative to control values.

FIG. 25: Analysis of the proportions of B cells and myeloid cells (NK, NKT and macrophages) within the peripheral blood of patients undergoing LHRH agonist treatment demonstrated a varying degree of change within subsets. While NK, NKT and macrophage proportions remained relatively constant following treatment, the proportion of B cells was decreased in 4/9 patients.

FIG. 26: Analysis of the total cell numbers of B and myeloid cells within the peripheral blood post-treatment showed clearly increased levels of NK (5/9 patients), NKT (4/9 patients) and macrophage (3/9 patients) cell numbers post-treatment. B cell numbers showed no distinct trend with 2/9 patients showing increased levels; 4/9 patients showing no change and 3/9 patients showing decreased levels.

FIG. 27: The major changes seen post-LHRH agonist treatment was within the T cell population of the peripheral blood. In particular there was a selective increase in the proportion of naïve (CD45RA+) CD4+ cells, with the ratio of naïve (CD45RA⁺) to memory (CD45RO⁺) in the CD4⁺ T cell subset increasing in 6/9 patients.

FIG. 28: Decrease in the impedance of skin using various laser pulse energies. There is a decrease in skin impedance in skin irradiated at energies as low as 10 mJ, using the fitted curve to interpolate data.

FIG. 29: Permeation of a pharmaceutical through skin. Permeability of the skin, using insulin as a sample pharmaceutical, was greatly increased through laser irradiation.

FIG. 30: Change in fluorescence of skin over time after the addition of 5-aminolevulenic acid (ALA) and a single impulse transient to the skin. The peak of intensity occurs at about 640 nm and is highest after 210 minutes (dashed line) post-treatment.

FIG. 31: Change in fluorescence of skin over time after the addition of 5-aminolevulenic acid (ALA) without an impulse transient. There is little change in the intensity at different time points.

FIG. 32: Comparison of change in fluorescence of skin after the addition of 5-aminolevulenic acid (ALA) and a single impulse transient under various peak stresses. The degree of permeabilization of the stratum corneum depends on the peak stress.

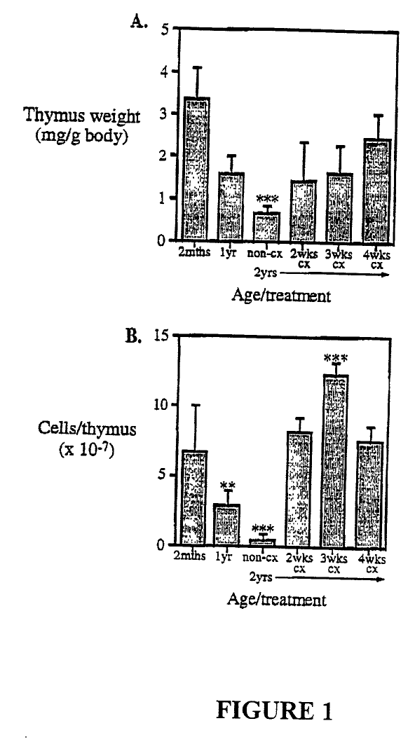

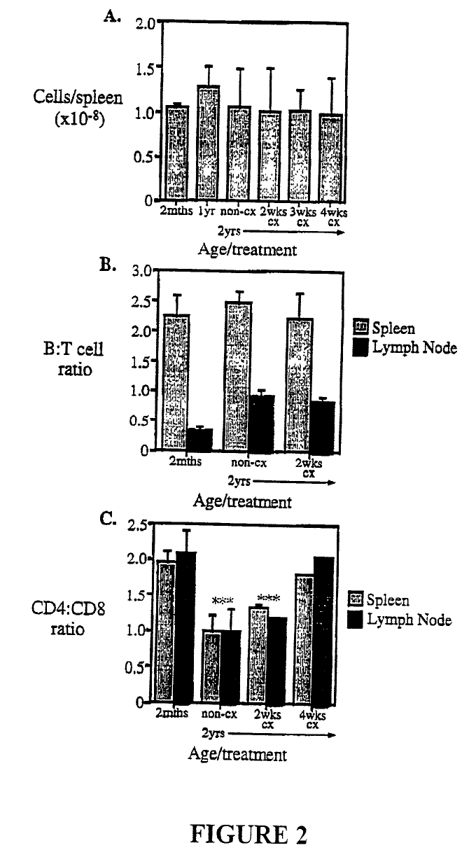

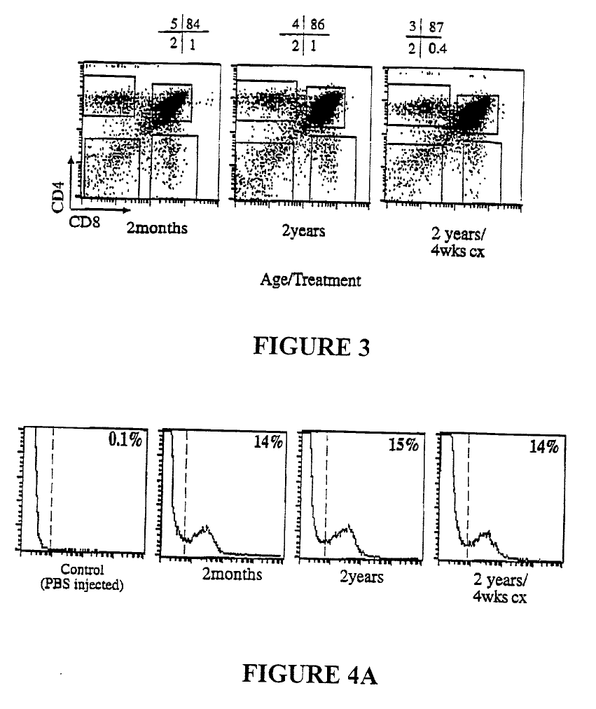

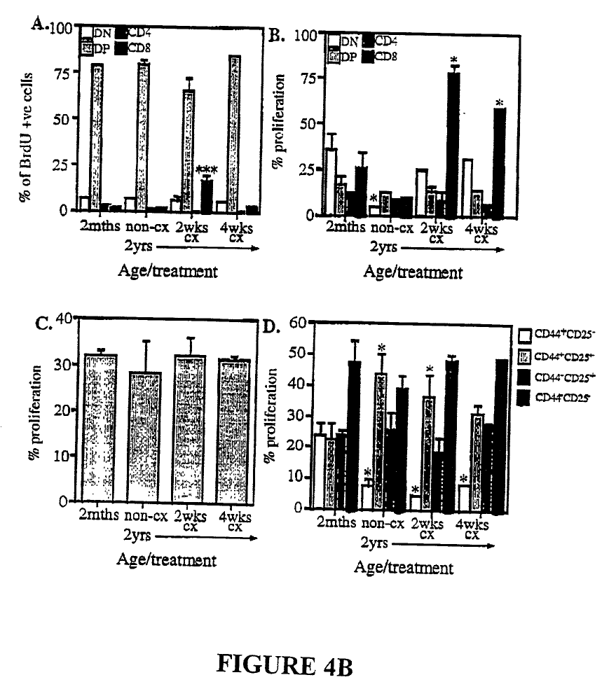

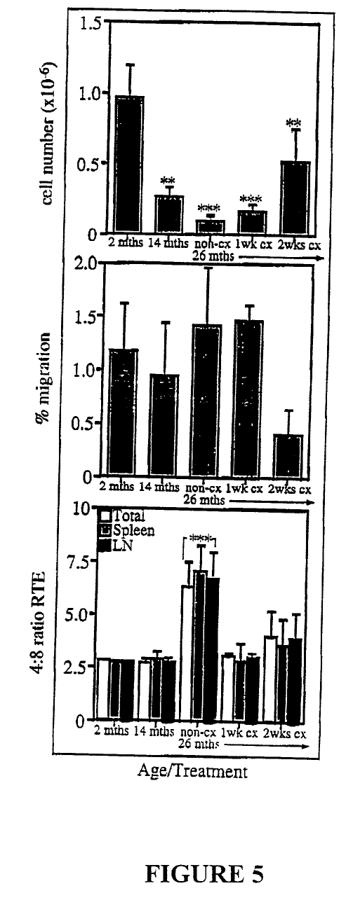

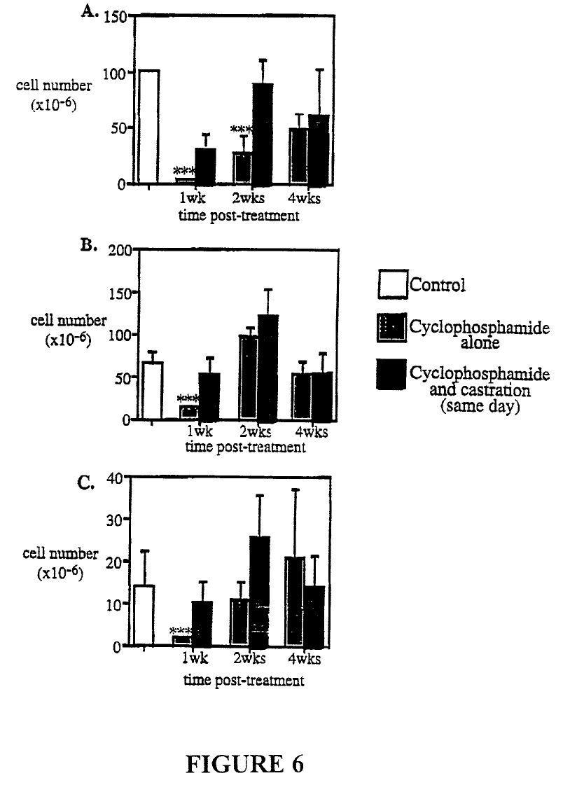

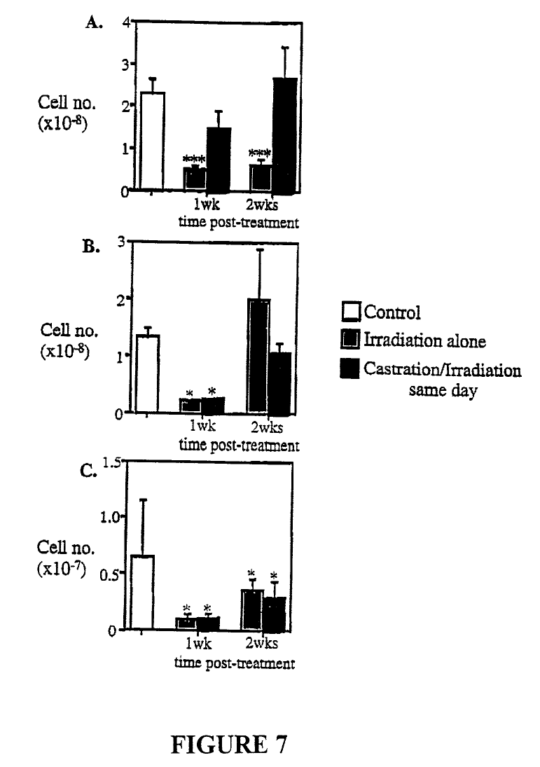

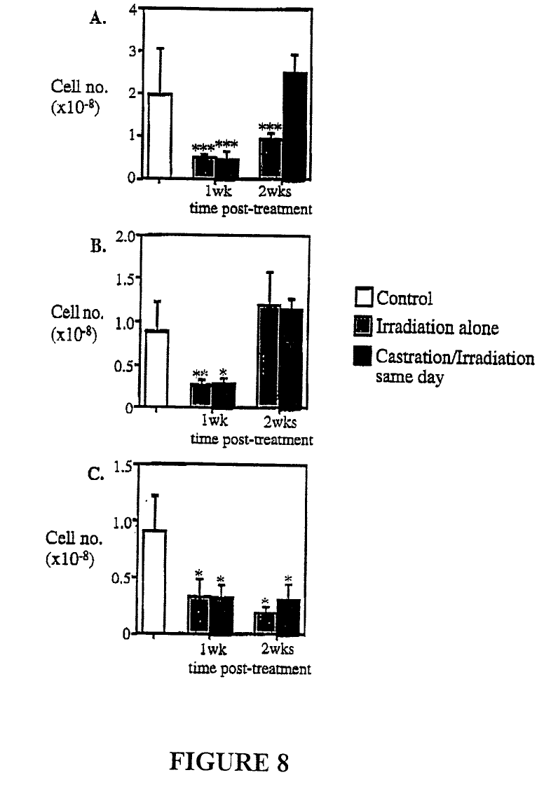

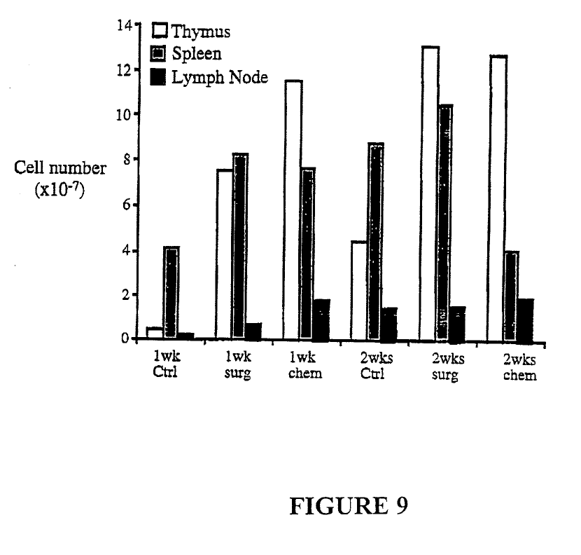

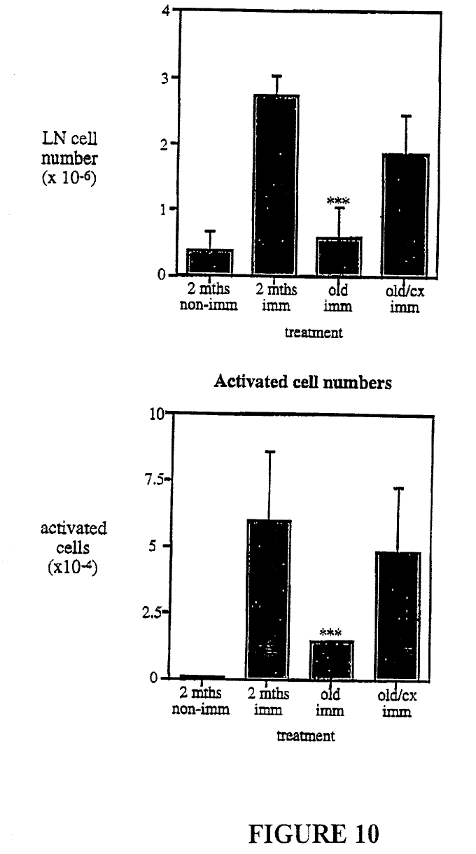

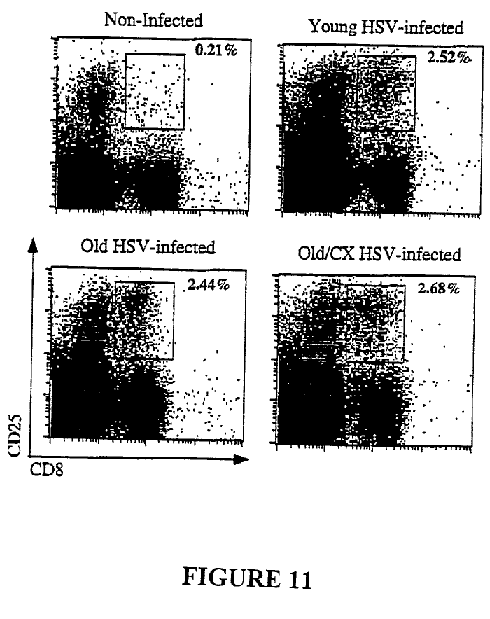

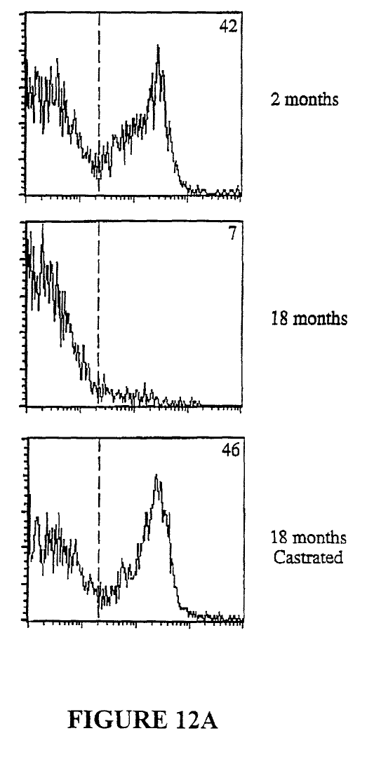

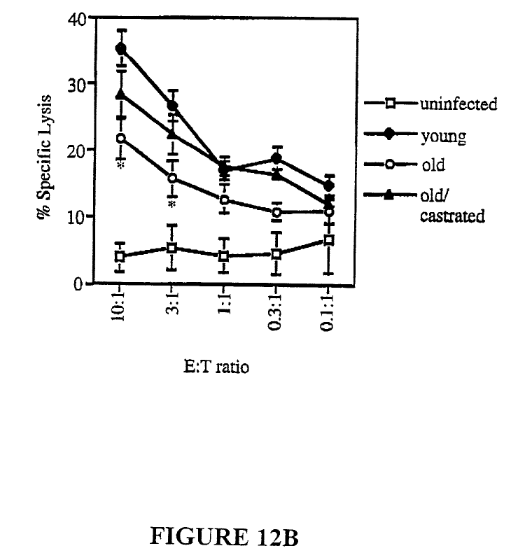

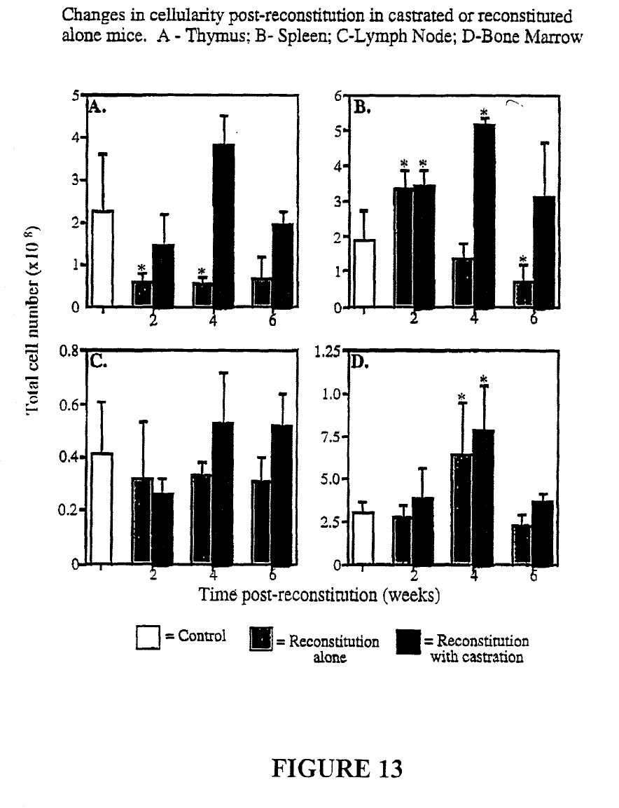

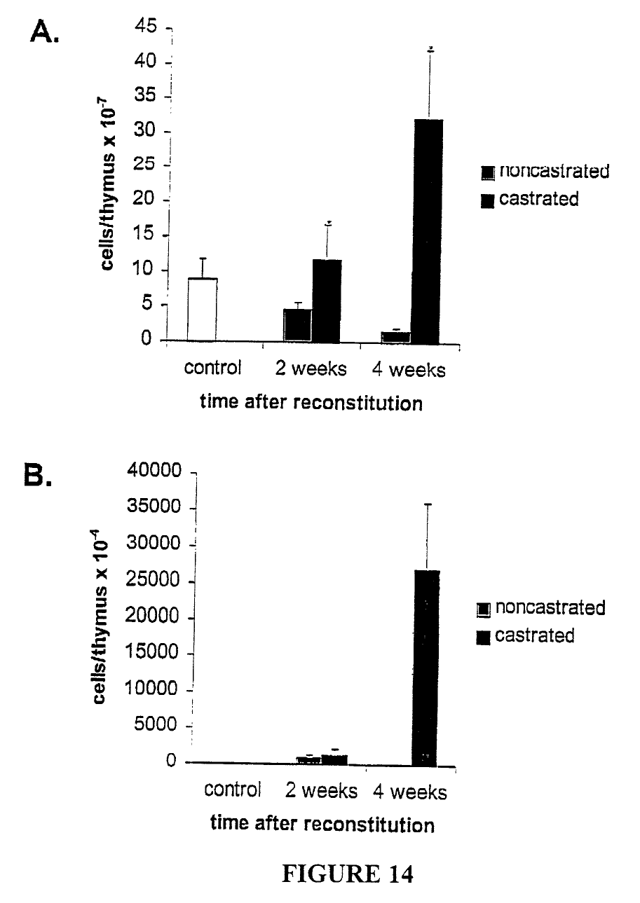

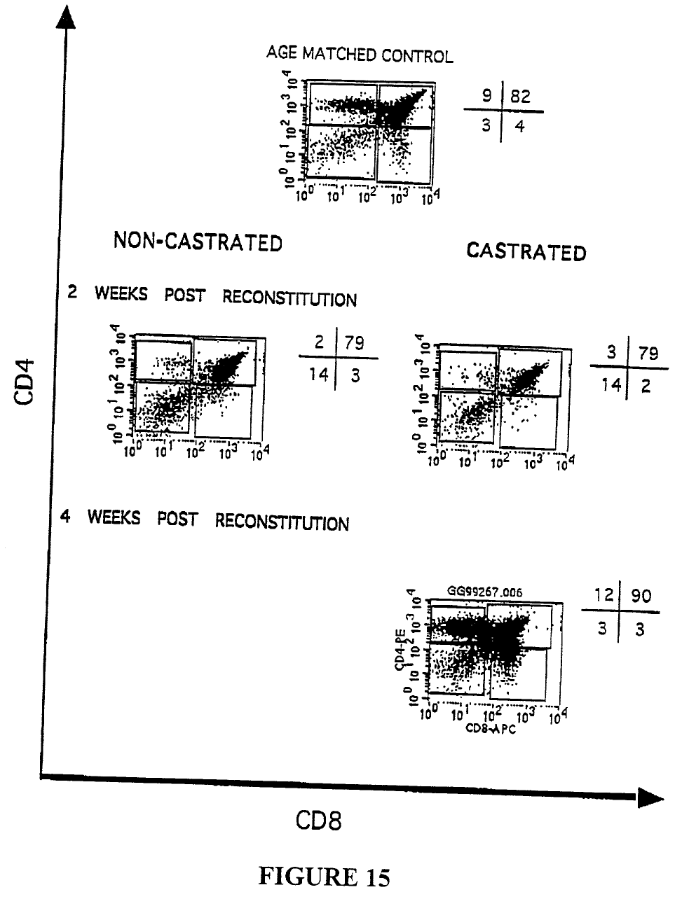

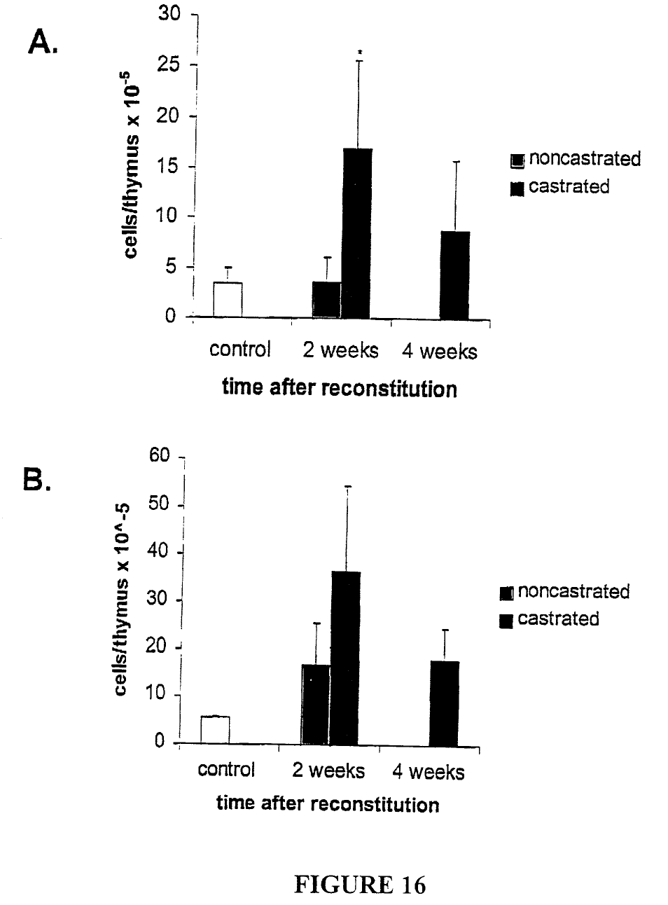

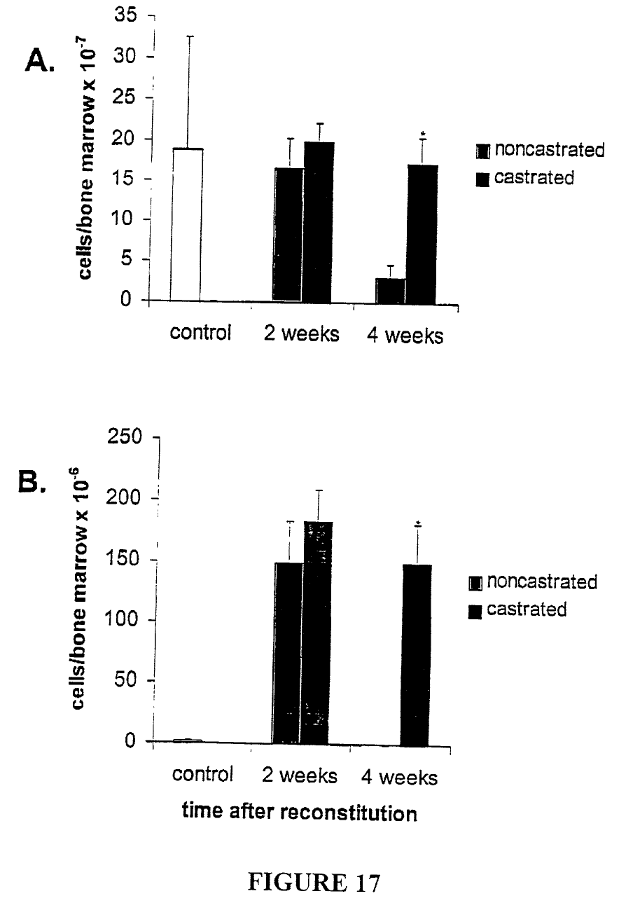

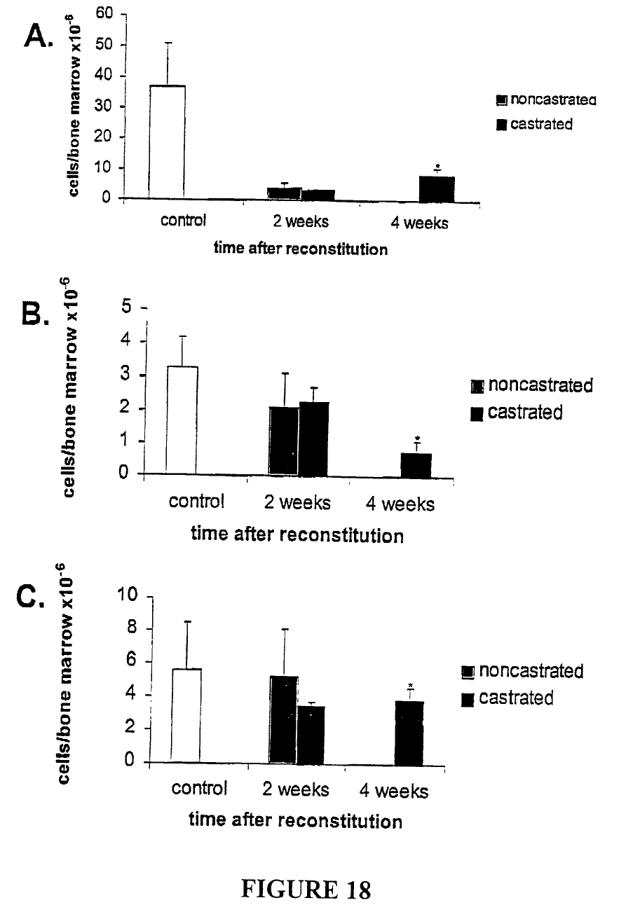

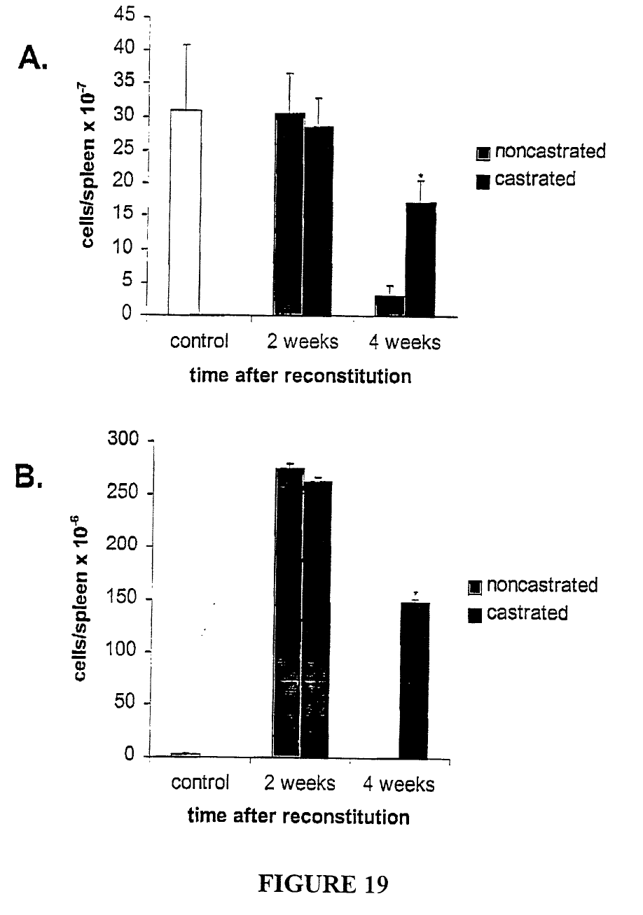

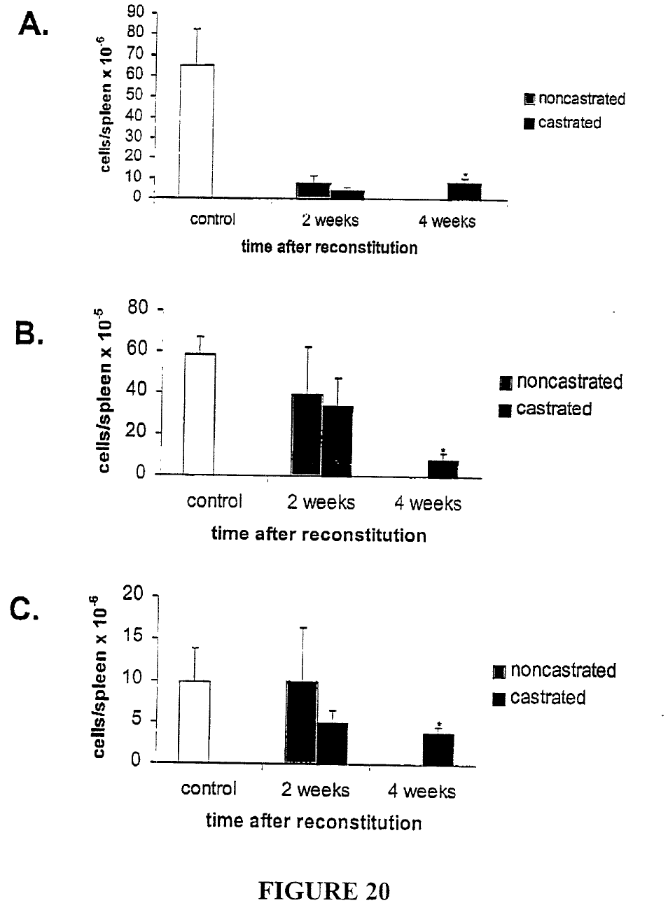

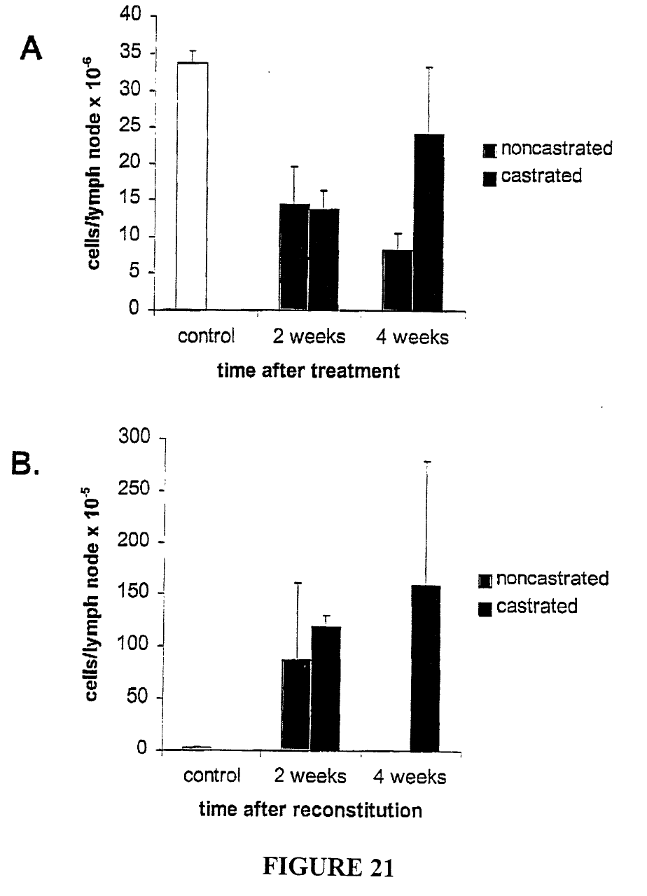

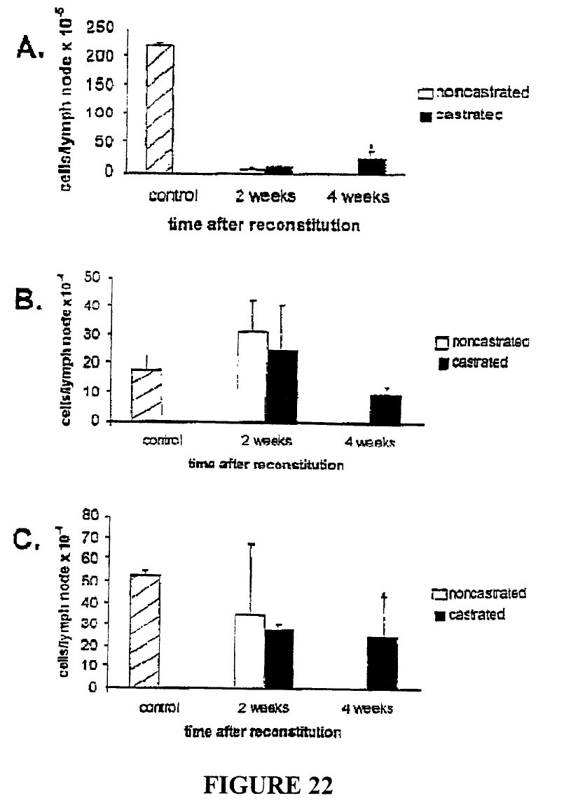

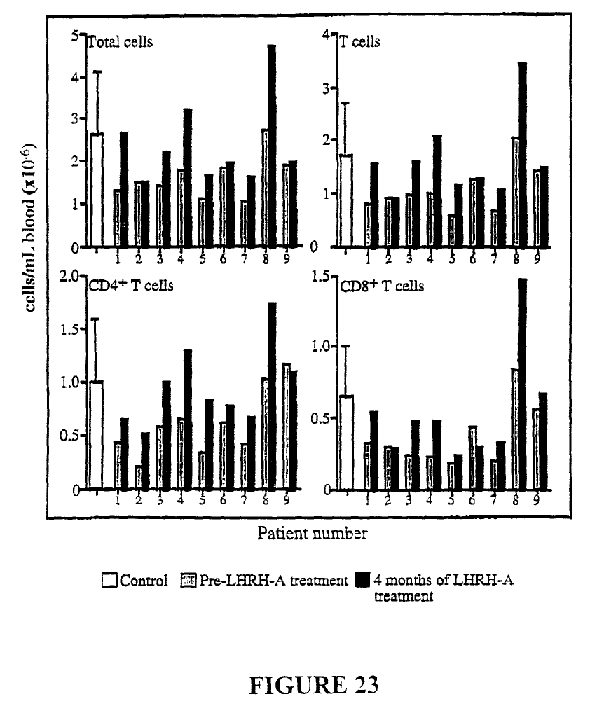

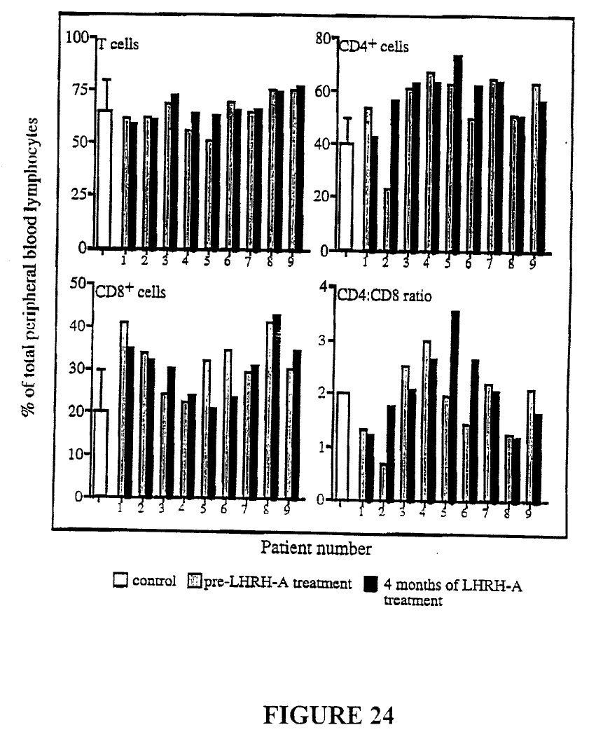

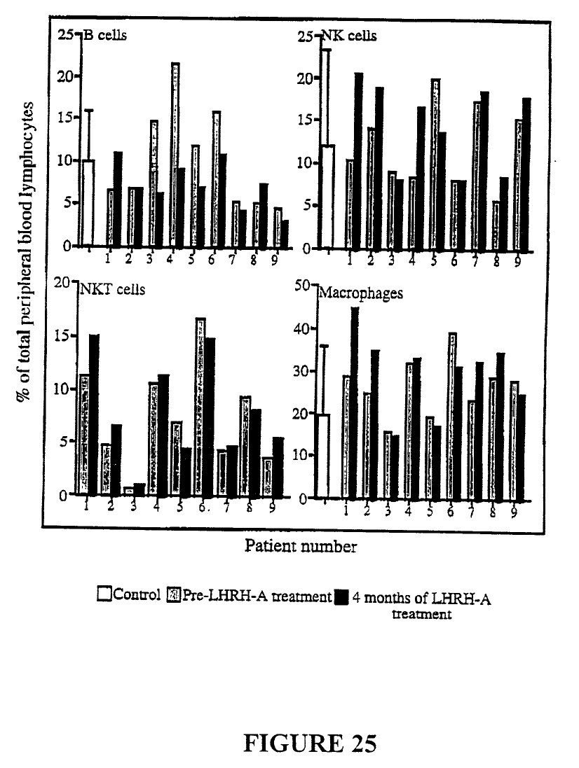

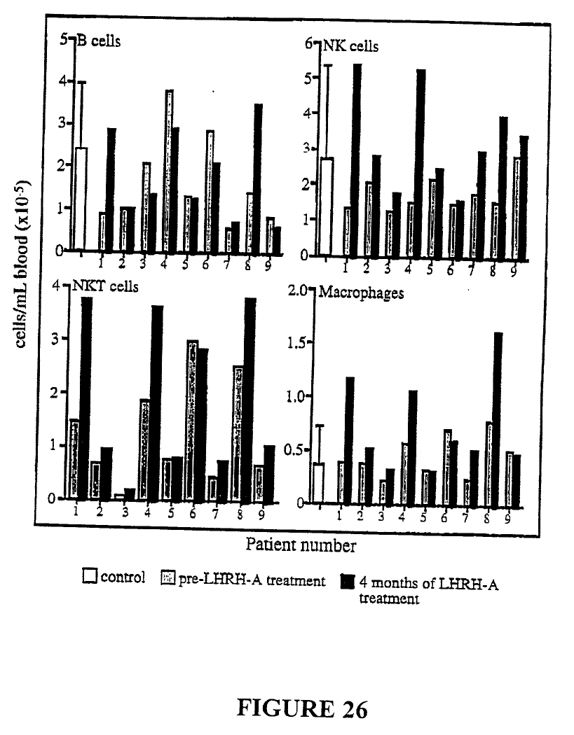

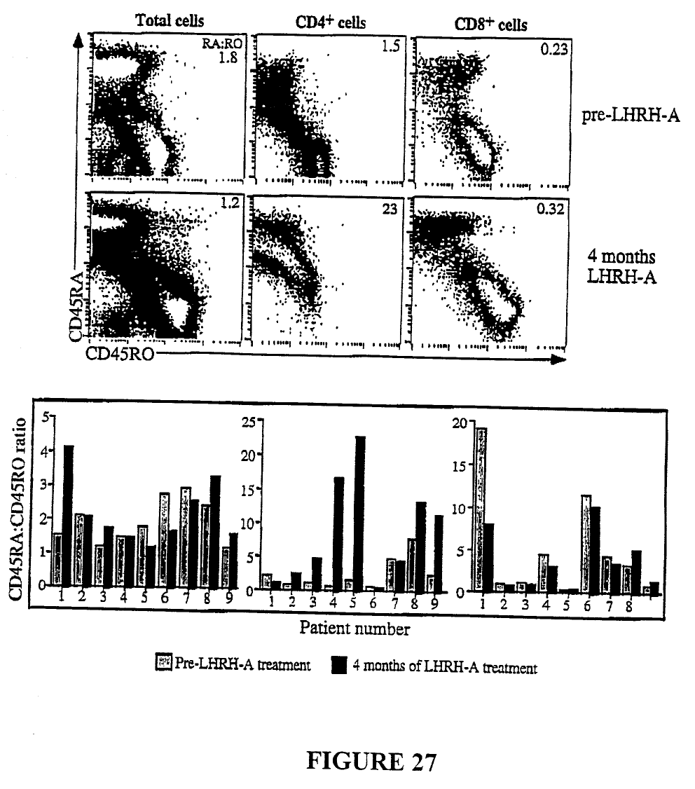

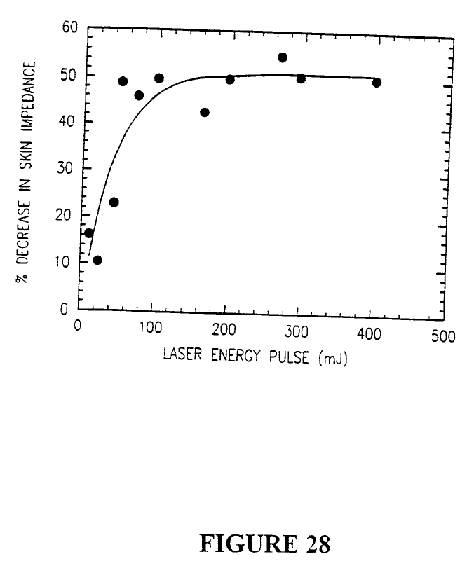

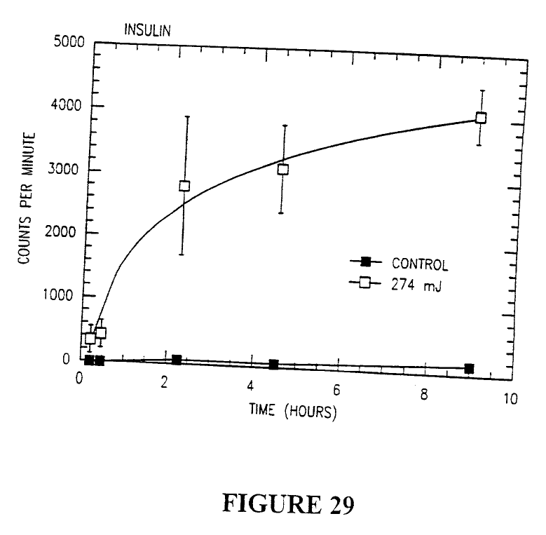

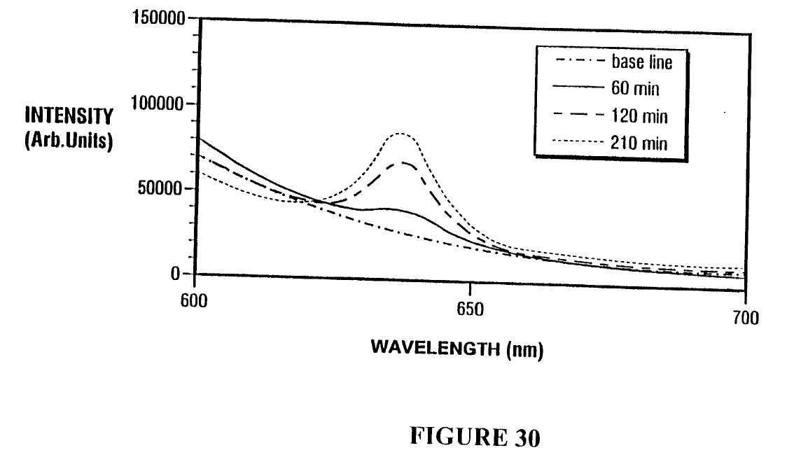

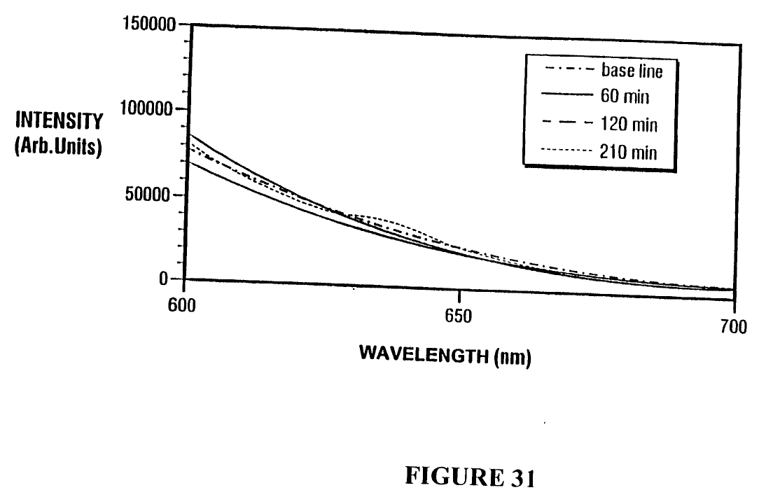

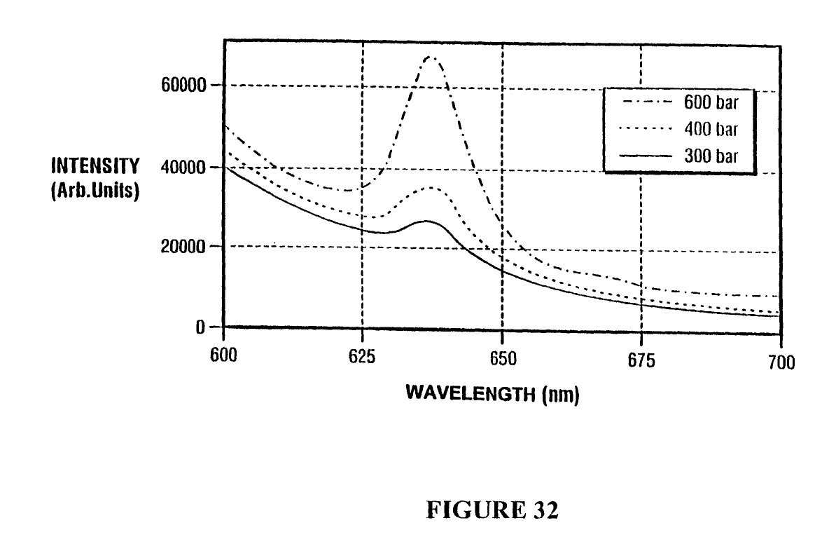

### DETAILED DESCRIPTION OF THE INVENTION

A characteristic feature of thymic function is that while it is of fundamental importance to the establishment and maintenance of the immune system and hence to the defense against infection and disease, it characteristically undergoes a profound age-dependent decrease in function to less than 5% of it maximal capacity. This becomes most pronounced following puberty, implicating a role for sex steroids.

Inhibition of sex steroids results, either directly or indirectly, in a major reactivation of thymic function, effectively reversing the atrophy. Given the broad range of patient age, diseases and treatments, however, it is anticipated that many patients will respond differently to this treatment, including some very poorly. Hence a new diagnostic early indicator of this responsiveness of the thymus to activation in the absence of sex steroids is provided to formulate rational clinical management of T cell based disorders.

Since the thymus is an endocrine organ, reactivation of thymic function involves release of not only new T cells into the blood stream after 2-4 weeks, but prior to this the thymus will also release increased levels of cytokines, even within hours of reactivation. These will be detectable in the blood or plasma. The present invention utilizes these released cells and molecules to detect the degree of response of a patient's thymus to inhibition of sex steroids. Provided here is a set of diagnostic techniques for making this determination.

Disruption of Sex Steroid Signaling to the Thymus

As will be readily understood, sex steroid signaling to the thymus can be disrupted in a range of ways well known to those of skill in the art, some of which are described herein. For example, inhibition of sex steroid production or blocking of one or more sex steroid receptors within the thymus will accomplish the desired disruption, as will administration of sex steroid agonists or antagonists, or active (antigen) or passive (antibody) anti-sex steroid vaccinations. Inhibition of sex steroid production can also be achieved by administration of one or more sex steroid analogs. In some clinical cases, permanent removal of the gonads via physical castration may be appropriate.

In a preferred embodiment the sex steroid signaling to the thymus is disrupted by administration of a sex steroid analog, preferably an analog of luteinizing hormone-releasing hormone (LHRH). Sex steroid analogs and their use in therapies and chemical castration are well known. Such analogs include, but are not limited to, Abarelix (U.S. Pat. No. 6,197,337), Cetrorelix, Deslorelin (described in U.S. Pat. No. 4,218,439), Eulexin (described in FR7923545, WO86/01105 and PT100899), Goserelin (described in U.S. Pat. No. 4,100,274, U.S. Pat. No. 4,128,638, GB9112859 and GB9112825), Leuprolide (described in U.S. Pat. No. 4,490,291, U.S. Pat. No. 3,972,859, U.S. Pat. No. 4,008,209, U.S. Pat. No. 4,005,063, DE2509783 and U.S. Pat. No. 4,992,421), Dioxalan derivatives such as are described in EP 413209, Triptorelin (described in U.S. Pat. No. 4,010,125, U.S. Pat. No. 4,018,726, U.S. Pat. No. 4,024,121, EP 364819 and U.S. Pat. No. 5,258,492), Meterelin (described in EP 23904), Buserelin (described in U.S. Pat. No. 4,003,884, U.S. Pat. No. 4,118,483 and U.S. Pat. No. 4,275,001), Histrelin (described in EP217659), Nafarelin (described in U.S. Pat. No. 4,234,571, WO93/15722 and EP52510), Lutrelin (described in U.S. Pat. No. 4,089,946), Leuprorelin (described in Plosker et al.) and LHRH analogs such as are described in EP181236, U.S. Pat. No. 4,608,251, U.S. Pat. No. 4,656,247, U.S. Pat. No. 4,642,332, U.S. Pat. No. 4,010,149, U.S. Pat. No. 3,992,365 and U.S. Pat. No. 4,010,149. The disclosures of each the references referred to above are incorporated herein by reference.

While the stimulus for thymic regeneration is fundamentally based on the inhibition of the effects of sex steroids and/or the direct effects of the LHRH analogues, it may be necessary to include additional substances which can act in concert to enhance the thymic effect. Such compounds could include but not be limited to Interleukin 7, members of the epithelium and fibroblast growth factor familes and keratinocyte growth factor. It is envisaged that these additional compounds would only be given once at the initial LHRH analogue application. In addition steroid receptor based modulators, which may be targeted to be thymic specific could be developed and used.

Delivery of Agents for Chemical Castration

Delivery of the compounds of this invention can be accomplished via a number of methods known to persons skilled in the art. One standard procedure for administering chemical inhibitors to inhibit sex steroid signaling to the thymus utilizes a single dose of an LHRH agonist that is effective for three months. For this a simple one-time i.v. or i.m. injection would not be sufficient as the agonist would be cleared from the patient's body well before the three months are over. Instead, a depot injection or an implant may be used, or any other means of delivery of the inhibitor that will allow slow release of the inhibitor. Likewise, a method for increasing the half life of the inhibitor within the body, such as by modification of the chemical, while retaining the function required herein, may be used.

Examples of more useful delivery mechanisms include, but are not limited to, laser irradiation of the skin, and creation of high pressure impulse transients (also called stress waves or impulse transients) on the skin, each method accompanied or followed by placement of the compound(s) with or without carrier at the same locus. A preferred method of this placement is in a patch placed and maintained on the skin for the duration of the treatment.

One means of delivery utilizes a laser beam, specifically focused, and lasing at an appropriate wavelength, to create small perforations or alterations in the skin of a patient. See U.S. Pat. No. 4,775,361, U.S. Pat. No. 5,643,252, U.S. Pat. No. 5,839,446, and U.S. Pat. No. 6,056,738, all of which are incorporated herein by reference. In a preferred embodiment, the laser beam has a wavelength between 0.2 and 10 microns. More preferably, the wavelength is between about 1.5 and 3.0 microns. Most preferably the wavelength is about 2.94 microns. In one embodiment, the laser beam is focused with a lens to produce an irradiation spot on the skin through the epidermis of the skin. In an additional embodiment, the laser beam is focused to create an irradiation spot only through the stratum corneum of the skin.

Several factors may be considered in defining the laser beam, including wavelength, energy fluence, pulse temporal width and irradiation spot-size. In a preferred embodiment, the energy fluence is in the range of 0.03-100,000 J/cm². More preferably, the energy fluence is in the range of 0.03-9.6 J/cm². The beam wavelength is dependent in part on the laser material, such as Er:YAG. The pulse temporal width is a consequence of the pulse width produced by, for example, a bank of capacitors, the flashlamp, and the laser rod material. The pulse width is optimally between 1 fs (femtosecond) and 1,000 μs.

According to this method the perforation or alteration produced by the laser need not be produced with a single pulse from the laser. In a preferred embodiment a perforation or alteration through the stratum corneum is produced by using multiple laser pulses, each of which perforates or alters only a fraction of the target tissue thickness.

To this end, one can roughly estimate the energy required to perforate or alter the stratum corneum with multiple pulses by taking the energy in a single pulse and dividing by the number of pulses desirable. For example, if a spot of a particular size requires 1 J of energy to produce a perforation or alteration through the entire stratum corneum, then one can produce qualitatively similar perforation or alteration using ten pulses, each having 1/10th the energy. Because it is desirable that the patient not move the target tissue during the irradiation (human reaction times are on the order of 100 ms or so), and that the heat produced during each pulse not significantly diffuse, in a preferred embodiment the pulse repetition rate from the laser should be such that complete perforation is produced in a time of less than 100 ms. Alternatively, the orientation of the target tissue and the laser can be mechanically fixed so that changes in the target location do not occur during the longer irradiation time.

To penetrate the skin in a manner that induces little or no blood flow, skin is perforated or altered through the outer surface, such as the stratum corneum layer, but not as deep as the capillary layer. The laser beam is focussed precisely on the skin, creating a beam diameter at the skin in the range of approximately 0.5 microns -5.0 cm. Optionally, the spot can be slit-shaped, with a width of about 0.05-0.5 mm and a length of up to 2.5 mm. The width can be of any size, being controlled by the anatomy of the area irradiated and the desired permeation rate of the fluid to be removed or the pharmaceutical to be applied. The focal length of the focusing lens can be of any length, but in one embodiment it is 30 mm.

By modifying wavelength, pulse length, energy fluence (which is a function of the laser energy output (in Joules) and size of the beam at the focal point (cm²)), and irradiation spot size, it is possible to vary the effect on the stratum corneum between ablation (perforation) and non-ablative modification (alteration). Both ablation and non-ablative alteration of the stratum corneum result in enhanced permeation of subsequently applied pharmaceuticals.

For example, by reducing the pulse energy while holding other variables constant, it is possible to change between ablative and non-ablative tissue-effect. Using an Er:YAG laser having a pulse length of about 300 μs, with a single pulse or radiant energy and irradiating a 2 mm spot on the skin, a pulse energy above approximately 100 mJ causes partial or complete ablation, while any pulse energy below approximately 100 mJ causes partial ablation or non-ablative alteration to the stratum corneum. Optionally, by using multiple pulses, the threshold pulse energy required to enhance permeation of body fluids or for pharmaceutical delivery is reduced by a factor approximately equal to the number of pulses.

Alternatively, by reducing the spot size while holding other variables constant, it is also possible to change between ablative and non-ablative tissue-effect. For example, halving the spot area will result in halving the energy required to produce the same effect. Irradiation down to 0.5 microns can be obtained, for example, by coupling the radiant output of the laser into the objective lens of a microscope objective. (e.g., as available from Nikon, Inc., Melville, N.Y.). In such a case, it is possible to focus the beam down to spots on the order of the limit of resolution of the microscope, which is perhaps on the order of about 0.5 microns. In fact, if the beam profile is Gaussian, the size of the affected irradiated area can be less than the measured beam size and can exceed the imaging resolution of the microscope. To non-ablatively alter tissue in this case, it would be suitable to use a 3.2 J/cm² energy fluence, which for a half-micron spot size would require a pulse energy of about 5 nJ. This low a pulse energy is readily available from diode lasers, and can also be obtained from, for example, the Er:YAG laser by attenuating the beam by an absorbing filter, such as glass.

Optionally, by changing the wavelength of radiant energy while holding the other variables constant, it is possible to change between an ablative and non-ablative tissue-effect. For example, using Ho:YAG (holmium: YAG; 2.127 microns) in place of the Er:YAG (erbium: YAG; 2.94 microns) laser, would result in less absorption of energy by the tissue, creating less of a perforation or alteration.

Picosecond and femtosecond pulses produced by lasers can also be used to produce alteration or ablation in skin. This can be accomplished with modulated diode or related microchip lasers, which deliver single pulses with temporal widths in the 1 femtosecond to 1 ms range. (See D. Stem et al., “Corneal Ablation by Nanosecond, Picosecond, and Femtosecond Lasers at 532 and 625 μm,” Corneal Laser Ablation, Vol. 107, pp. 587-592 (1989), incorporated herein by reference, which discloses the use of pulse lengths down to 1 femtosecond).

Another delivery method uses high pressure impulse transients on skin to create permeability. See U.S. Pat. No. 5,614,502, and U.S. Pat. No. 5,658,892, both of which are incorporated herein by reference. High pressure impulse transients, e.g., stress waves (e.g., laser stress waves (LSW) when generated by a laser), with specific rise times and peak stresses (or pressures), can safely and efficiently effect the transport of compounds, such as those of the present invention, through layers of epithelial tissues, such as the stratum corneum and mucosal membranes. These methods can be used to deliver compounds of a wide range of sizes regardless of their net charge. In addition, impulse transients used in the present methods avoid tissue injury.

Prior to exposure to an impulse transient, an epithelial tissue layer, e.g., the stratum corneum, is likely impermeable to a foreign compound; this prevents diffusion of the compound into cells underlying the epithelial layer. Exposure of the epithelial layer to the impulse transients enables the compound to diffuse through the epithelial layer. The rate of diffusion, in general, is dictated by the nature of the impulse transients and the size of the compound to be delivered.

The rate of penetration through specific epithelial tissue layers, such as the stratum corneum of the skin, also depends on several other factors including pH, the metabolism of the cutaneous substrate tissue, pressure differences between the region external to the stratum corneum, and the region internal to the stratum corneum, as well as the anatomical site and physical condition of the skin. In turn, the physical condition of the skin depends on health, age, sex, race, skin care, and history. For example, prior contacts with organic solvents or surfactants affect the physical condition of the skin.

The amount of compound delivered through the epithelial tissue layer will also depend on the length of time the epithelial layer remains permeable, and the size of the surface area of the epithelial layer which is made permeable. The properties and characteristics of impulse transients are controlled by the energy source used to create them. See WO 98/23325, which is incorporated herein by reference. However, their characteristics are modified by the linear and non-linear properties of the coupling medium through which they propagate. The linear attenuation caused by the coupling medium attenuates predominantly the high frequency components of the impulse transients. This causes the bandwidth to decrease with a corresponding increase in the rise time of the impulse transient. The non-linear properties of the coupling medium, on the other hand, cause the rise time to decrease. The decrease of the rise time is the result of the dependence of the sound and particle velocity on stress (pressure). As the stress increases, the sound and the particle velocity increase as well. This causes the leading edge of the impulse transient to become steeper. The relative strengths of the linear attenuation, non-linear coefficient, and the peak stress determine how long the wave has to travel for the increase in steepness of rise time to become substantial.

The rise time, magnitude, and duration of the impulse transient are chosen to create a non-destructive (i.e., non-shock wave) impulse transient that temporarily increases the permeability of the epithelial tissue layer. Generally the rise time is at least 1 ns, and is more preferably about 10 ns.

The peak stress or pressure of the impulse transients varies for different epithelial tissue or cell layers. For example, to transport compounds through the stratum corneum, the peak stress or pressure of the impulse transient should be set to at least 400 bar; more preferably at least 1,000 bar, but no more than about 2,000 bar.

For epithelial mucosal layers, the peak pressure should be set to between 300 bar and 800 bar, and is preferably between 300 bar and 600 bar.

The impulse transients preferably have durations on the order of a few tens of ns, and thus interact with the epithelial tissue for only a short period of time.

Following interaction with the impulse transient, the epithelial tissue is not permanently damaged, but remains permeable for up to about three minutes.

In addition, the new methods involve the application of only a few discrete high amplitude pulses to the patient. The number of impulse transients administered to the patient is typically less than 100, more preferably less than 50, and most preferably less than 10. When multiple optical pulses are used to generate the impulse transient, the time duration between sequential pulses is 10 to 120 seconds, which is long enough to prevent permanent damage to the epithelial tissue.

Properties of impulse transients can be measured using methods standard in the art. For example, peak stress or pressure, and rise time can be measured using a polyvinylidene fluoride (PVDF) transducer method as described in Doukas et al., Ultrasound Med. Biol., 21:961 (1995).

Impulse transients can be generated by various energy sources. The physical phenomenon responsible for launching the impulse transient is, in general, chosen from three different mechanisms: (1) thermoelastic generation; (2) optical breakdown; or (3) ablation.

For example, the impulse transients can be initiated by applying a high energy laser source to ablate a target material, and the impulse transient is then coupled to an epithelial tissue or cell layer by a coupling medium. The coupling medium can be, for example, a liquid or a gel, as long as it is non-linear. Thus, water, oil such as castor oil, an isotonic medium such as phosphate buffered saline (PBS), or a gel such as a collagenous gel, can be used as the coupling medium.

In addition, the coupling medium can include a surfactant that enhances transport, e.g., by prolonging the period of time in which the stratum corneum remains permeable to the compound following the generation of an impulse transient. The surfactant can be, e.g., ionic detergents or nonionic detergents and thus can include, e.g., sodium lauryl sulfate, cetyl trimethyl ammonium bromide, and lauryl dimethyl amine oxide.

The absorbing target material acts as an optically triggered transducer. Following absorption of light, the target material undergoes rapid thermal expansion, or is ablated, to launch an impulse transient. Typically, metal and polymer films have high absorption coefficients in the visible and ultraviolet spectral regions.

Many types of materials can be used as the target material in conjunction with a laser beam, provided they fully absorb light at the wavelength of the laser used. The target material can be composed of a metal such as aluminum or copper; a plastic, such as polystyrene, e.g., black polystyrene; a ceramic; or a highly concentrated dye solution. The target material must have dimensions larger than the cross-sectional area of the applied laser energy. In addition, the target material must be thicker than the optical penetration depth so that no light strikes the surface of the skin. The target material must also be sufficiently thick to provide mechanical support. When the target material is made of a metal, the typical thickness will be 1/32 to 1/16 inch. For plastic target materials, the thickness will be 1/16 to ⅛ inch.

Impulse transients can be also enhanced using confined ablation. In confined ablation, a laser beam transparent material, such as a quartz optical window, is placed in close contact with the target material. Confinement of the plasma, created by ablating the target material by using the transparent material, increases the coupling coefficient by an order of magnitude (Fabro et al., J. Appl. Phys., 68:775, 1990). The transparent material can be quartz, glass, or transparent plastic.

Since voids between the target material and the confining transparent material allow the plasma to expand, and thus decrease the momentum imparted to the target, the transparent material is preferably bonded to the target material using an initially liquid adhesive, such as carbon-containing epoxies, to prevent such voids.

The laser beam can be generated by standard optical modulation techniques known in the art, such as by employing Q-switched or mode-locked lasers using, for example, electro- or acousto-optic devices. Standard commercially available lasers that can operate in a pulsed mode in the infrared, visible, and/or infrared spectrum include Nd:YAG, Nd:YLF, CO₂, excimer, dye, Ti:sapphire, diode, holmium (and other rare-earth materials), and metal-vapor lasers. The pulse widths of these light sources are adjustable, and can vary from several tens of picoseconds (ps) to several hundred microseconds. For use in the new methods, the optical pulse width can vary from 100 ps to about 200 ns and is preferably between about 500 ps and 40 ns.

Impulse transients can also be generated by extracorporeal lithotripters (one example is described in Coleman et al., Ultrasound Med. Biol., 15:213-227, 1989). These impulse transients have rise times of 30 to 450 ns, which is longer than laser-generated impulse transients. To form an impulse transient of the appropriate rise time for the new methods using an extracorporeal lithotripter, the impulse transient is propagated in a non-linear coupling medium (e.g., water) for a distance determined by equation (1), above. For example, when using a lithotripter creating an impulse transient having a rise time of 100 ns and a peak pressure of 500 barr, the distance that the impulse transient should travel through the coupling medium before contacting an epithelial cell layer is approximately 5 mm.

An additional advantage of this approach for shaping impulse transients generated by lithotripters is that the tensile component of the wave will be broadened and attenuated as a result of propagating through the non-linear coupling medium. This propagation distance should be adjusted to produce an impulse transient having a tensile component that has a pressure of only about 5 to 10% of the peak pressure of the compressive component of the wave. Thus, the shaped impulse transient will not damage tissue.

The type of lithotripter used is not critical. Either an electrohydraulic, electromagnetic, or piezoelectric lithotripter can be used.

The impulse transients can also be generated using transducers, such as piezoelectric transducers. Preferably, the transducer is in direct contact with the coupling medium, and undergoes rapid displacement following application of an optical, thermal, or electric field to generate the impulse transient. For example, dielectric breakdown can be used, and is typically induced by a high-voltage spark or piezoelectric transducer (similar to those used in certain extracorporeal lithotripters, Coleman et al., Ultrasound Med. Biol., 15:213-227, 1989). In the case of a piezoelectric transducer, the transducer undergoes rapid expansion following application of an electrical field to cause a rapid displacement in the coupling medium.

In addition, impulse transients can be generated with the aid of fiber optics. Fiber optic delivery systems are particularly maneuverable and can be used to irradiate target materials located adjacent to epithelial tissue layers to generate impulse transients in hard-to reach places. These types of delivery systems, when optically coupled to lasers, are preferred as they can be integrated into catheters and related flexible devices, and used to irradiate most organs in the human body. In addition, to launch an impulse transient having the desired rise times and peak stress, the wavelength of the optical source can be easily tailored to generate the appropriate absorption in a particular target material.

Alternatively, an energetic material can produce an impulse transient in response to a detonating impulse. The detonator can detonate the energetic material by causing an electrical discharge or spark.

Hydrostatic pressure can be used in conjunction with impulse transients to enhance the transport of a compound through the epithelial tissue layer. Since the effects induced by the impulse transients last for several minutes, the transport rate of a drug diffusing passively through the epithelial cell layer along its concentration gradient can be increased by applying hydrostatic pressure on the surface of the epithelial tissue layer, e.g., the stratum corneum of the skin, following application of the impulse transient.

Diagnostic Indicators of Thymic Function

A. Known Markers

Certain markers are associated with the activation of the thymus. By following the concentration of any one, or any combination, of these markers, one can monitor the level of activation of the thymus. Changes in the levels of these marker molecules pre-and post-activation of thymic function can be examined using bioinformatics. For example, two-dimensional gel electrophoresis of plasma (i.e., blood depleted of all cells by centrifugation) is performed on patients' samples pre- and post-inhibition of sex steroids. The differentially expressed “dots” on the gels are recorded and analyzed by computer.

1. Interleukin-7 (Il-7)

The major lymphopoietic and thymopoietic cytokine produced by thymic cortical epithelial cells, IL-7 is essential for the proliferation and differentiation of immature thymocytes (von Freeden-Jeffry et al., 1995; Komschlies et al., 1995; Peschon et al., 1994). Triple negative cell development requires interaction with IL-7 (Oosterwegel et al., 1997; Moore et al., 1993), which acts primarily by inducing bcl-2 expression and inhibiting programmed cell death of immature thymocytes (Akashi et al., 1997; Maraskovsky et al., 1997). Treatment with IL-7 alone has been demonstrated to reverse both the increase in apoptosis and decline in thymopoiesis within the CD44⁺CD25⁺ (TN2) and CD44⁻CD25⁺ (TN3) subsets, corresponding to the location of TCR β-chain rearrangement, in aged mice (Andrew & Aspinall, 2001).

Immune recovery in mice after T cell-depleted bone marrow transplantation has been documented to be enhanced following administration of IL-7, suggesting the production of IL-7 may be one of the mechanisms regulating de novo production of T cells after bone marrow transplantation (Bolotin et al., 1996). Analysis of IL-7 serum levels in patients before and after bone marrow transplantation by ELISA revealed an inverse relationship to absolute lymphocyte count (Bolotin et al., 1999). Studies measuring IL-7 levels in HIV-infected pediatric and adult patients also indicate a strong inverse correlation between IL-7 and absolute CD4 counts and lesser but significant correlations with CD3 and CD8 counts (Fry et al., 2001).

The mechanism underlying the increase in circulating IL-7 are unclear but it has been suggested that decreased T cell numbers result in diminished IL-7 receptor availability leading to increased levels of free IL-7 with no change in IL-7 production. That is, binding to lymphocytes that express IL-7 receptors (Bolotin et al., 1999) homeostatically regulates circulating IL-7 levels. An alternative mechanism is the direct upregulation of IL-7 in response to lymphopoenia through the interaction of T cells and IL-7-producing cells via a soluble mediator or through direct contact within the lymphoid microenvironment (Fry et al., 2001).

Normal IL-7 Levels

In children aged 6-months to 5.5 years, the normal mean concentration of IL-7 is 10.7±3.9 pg/ml. In adults aged 22.2 to 53.5 years the mean is appreciably lower, at 3.1±2.5 pg/ml. It has thus been suggested that IL-7 levels may be determined by age since IL-7 levels are highest in infants less than one year of age and lower in children and adults (Bolotin et al., 1999). This would support previous studies which demonstrated an age-dependent decline in thymopoietic capacity in chemotherapy and bone marrow transplant patients beginning in adolescence (Mackall et al., 1995; Weinberg et al., 1995). Moreover studies of bone marrow stroma from aged mice have shown decreased secretion of IL-7 with age (Stephan et al., 1998).

According to an embodiment of the present invention, concentration of IL-7 in a patient's blood or serum is monitored before and after administration of the agent(s) that block sex steroid mediated signaling to the thymus. Rise in the concentration of IL-7 within 2-3 days, preferably within 24 hours, more preferably within 2-3 hours, of administration of the agent(s) signifies that the thymus is responding to blockage of the sex steroid activity. Concentration of IL-7 is periodically monitored to determine the level of activation of the thymus over time.

2. Facteur Thymique Serique (FTS)

It is now largely established that the immune and neuroendocrine systems cross-talk by using similar ligands and receptors. The thymic-hypothalamus/pituitary axis constitutes a bi-directional circuit where the ascending feedback loop is effected by thymic factors of epithelial origin. Aside from modulating the release of peptidic hormones and neuropeptides, thymic hormones act mainly to promote the phenotypic maturation of progenitor cells from the bone marrow and to modulate mature T cell function (Ritter and Crispe, 1992). Hence thymic hormones may be important in a large spectrum of pathological conditions ranging from immunodeficiencies to neuroendocrine diseases.

FTS or thymulin is a nonapeptide hormone secreted exclusively by the thymic subcapsular and medullary cells (Ritter and Crispe, 1992). Essential for both early and late stages of T cell differentiation as well as T cell function, FTS also induces expression of several T cell markers, and promotes T cell functions such as allogeneic cytotoxicity, suppressor functions and IL-2 production (Ritter and Crispe, 1992).

FTS titers in children gradually increase with increasing age from 2.69±1.10 at a few days of age to 4.77±0.44 at a few years of age, then gradually decrease to 0.66±0.26 at 36 years of age to old age (Consolini et al., 2000). As the thymus is physiologically under neuroendocrine control, peptide hormones and neuropeptides influence age-related fluctuations in FTS levels. As noted above, impaired hormonal activity has been shown to be associated with age-related thymic atrophy (Consoloni et al., 2000). In particular, thymic atrophy is most evident following the rise in serum sex steroid levels following puberty (Fabris et al., 1997). Moreover FTS secretion by thymic epithelial cells is enhanced by growth hormone (Mocchegiani et al., 1990).

Zinc has been shown to be important in cellular immunity (Prasad et al., 1988), which is not surprising since FTS is biologically activated upon binding one molecule of zinc (Zn-FTS) (Bach, 1983). As zinc turnover is usually reduced with age (Panerai and Sacerdote, 1997), it has been postulated that the low FTS levels in old age can be related to a zinc deficiency (Mocchegiani and Fabris, 1995). Indeed it was found zinc treatment in elderly patients restores thymic secretory activity (Morcchegiani et al., 1990). However, in vitro studies on addition of zinc ions to plasma from adolescent patients did not restore the biological activity of FTS, indicating that the decreased FTS levels in adolescence is more likely related to the decline of thymic activity than zinc deficiency (Consolini et al., 2000).

In an embodiment of the present invention, the concentration of FTS in a patient's blood or serum is monitored before and after administration of the agent(s) that block sex steroid mediated signaling to the thymus. Rise in the concentration of FTS within 2-3 days, preferably within 24 hours, more preferably within 2-3 hours, of administration of the agent(s) signifies that the thymus is responding to blockage of the sex steroid activity. Concentration of FTS is periodically monitored to determine the level of activation of the thymus over time.

3. Thymosin and Thymopoietin

In contrast to FTS which begins to decline after 20 years of age in humans, thymosin-alpha 1 and thymopietin serum levels seem to decline as early as 10 years of age (reviewed in Bodey et al., 1997). Castration appears to increase thymosin-alpha 1 and thymosin-beta 4 serum levels as found in male rats (Windmill and Lee, 1999).

In an embodiment of the invention, the concentration of thymopoietin, thymosin-alpha 1, thymosin-beta 4, or combinations thereof are measured before and after administration of the agent(s) that block sex steroid mediated signaling to the thymus. Rise in the concentration of any of these compounds or combinations within 2-3 days, preferably within 24 hours, more preferably within 2-3 hours of administration of the agent(s) signifies that the thymus is responding to blockage of the sex steroid activity. Concentration of any of these compounds or combinations is periodically monitored to determine the level of activation of the thymus over time.

B. Newly Identified Markers

In addition to the known markers for thymic activation, several additional markers have been identified and used, based on the methods of the present invention.

Procedures for obtaining these markers can mimic those for following the already identified markers. For example, 2D gel electrophoresis can be used and the intensity of the various spots monitored over time. The spots will usually correspond to individual proteins, although occasionally there may be overlap or concurrence of spots from two or more different proteins. The identity of the molecules is revealed by solid phase amino acid sequencing. A new molecule(s) so identified as being altered in expression (increase or decrease) as a result of thymic activation will form the basis of a new diagnostic test for thymic responsiveness to loss of sex steroids.

T Cell Analysis

Monitoring of T cell production is another method that may be used to determine activation of the thymus. Techniques such as flow cytometric analysis of whole peripheral blood, detection of proliferating cells by monitoring the marker Ki67, and TREC analysis are among the methods known to those of skill in the field for such monitoring. In an embodiment of the invention, numbers of T cells, as well as proliferating T cells, are determined before and after administration of the agent(s) that block sex steroid mediated signaling to the thymus. Rise in the number of any of these T cells or combinations within 2-3 days, preferably within 24 hours, more preferably within 2-3 hours of administration of the agent(s) signifies that the thymus is responding to blockage of the sex steroid activity. Concentration of any of these T cells or combinations is periodically monitored to determine the level of activation of the thymus over time.

### EXAMPLES

### Example 1

### T Cell Depletion

In order to remove the abnormal T cells, the patient underwent T cell depletion. One standard procedure for this step is as follows: The human patient received anti-T cell antibodies in the form of a daily injection of 15 mg/kg of Atgam (xeno anti-T cell globulin, Pharmacia Upjohn) for a period of 10 days in combination with an inhibitor of T cell activation, cyclosporin A, 3 mg/kg, as a continuous infusion for 3-4 weeks followed by daily tablets at 9 mg/kg as needed. This treatment did not affect early T cell development in the patient's thymus, as the amount of antibody necessary to have such an affect cannot be delivered due to the size and configuration of the human thymus. The treatment was maintained for approximately 4-6 weeks to allow the loss of sex steroids followed by the reconstitution of the thymus. The prevention of T cell reactivity may also be combined with inhibitors of second level signals such as interleukins or cell adhesion molecules to enhance the T cell ablation.

Because in many cases it is not possible to reduce only the antigen-specific T cells which cause the disease, the whole population of T cells, including the pathological ones, is depleted. This depletion of peripheral T cells markedly retards the disease. Simultaneously, however, because of the lack of T cells, it induces a state of generalized immunodeficiency which means the patients are highly susceptible to infection, particularly viral. Even B cell responses will not function normally in the absence of appropriate T cell help.

### Example 2

### Sex Steroid Ablation Therapy

The patient was given sex steroid ablation therapy in the form of delivery of an LHRH agonist. This was given in the form of either Leucrin (depot injection; 22.5 mg) or Zoladex (implant; 10.8 mg), either one as a single dose effective for 3 months. This was effective in reducing sex steroid levels sufficiently to reactivate the thymus. In some cases it is also necessary to deliver a suppresser of adrenal gland production of sex steroids, such as Cosudex (5 mg/day) as one tablet per day for the duration of the sex steroid ablation therapy. Adrenal gland production of sex steroids makes up around 10-15% of a human's steroids.

Reduction of sex steroids in the blood to minimal values took about 1-3 weeks; concordant with this was the reactivation of the thymus. In some cases it is necessary to extend the treatment to a second 3 month injection/implant.

### Example 3

### Alternative Delivery Method

In place of the 3 month depot or implant administration of the LHRH agonist, alternative methods can be used. In one example the patient's skin may be irradiated by a laser such as an Er:YAG laser, to ablate or alter the skin so as to reduce the impeding effect of the stratum corneum.

A. Laser Ablation or Alteration: An infrared laser radiation pulse was formed using a solid state, pulsed, Er:YAG laser consisting of two flat resonator mirrors, an Er:YAG crystal as an active medium, a power supply, and a means of focusing the laser beam. The wavelength of the laser beam was 2.94 microns. Single pulses were used.

The operating parameters were as follows: The energy per pulse was 40, 80 or 120 mJ, with the size of the beam at the focal point being 2 mm, creating an energy fluence of 1.27, 2.55 or 3.82 J/cm². The pulse temporal width was 300 μs, creating an energy fluence rate of 0.42, 0.85 or 1.27×10⁴ W/cm².

Subsequently, an amount of LHRH agonist is applied to the skin and spread over the irradiation site. The LHRH agonist may be in the form of an ointment so that it remains on the site of irradiation. Optionally, an occlusive patch is placed over the agonist in order to keep it in place over the irradiation site.

Optionally a beam splitter is employed to split the laser beam and create multiple sites of ablation or alteration. This provides a faster flow of LHRH agonist through the skin into the blood stream. The number of sites can be predetermined to allow for maintenance of the agonist within the patient's system for the requisite period of time.

B. Pressure Wave: A dose of LHRH agonist is placed on the skin in a suitable container, such as a plastic flexible washer (about 1 inch in diameter and about 1/16 inch thick), at the site where the pressure wave is to be created. The site is then covered with target material such as a black polystyrene sheet about 1 mm thick. A Q-switched solid state ruby laser (20 ns pulse duration, capable of generating up to 2 joules per pulse) is used to generate the laser beam, which hits the target material and generates a single impulse transient. The black polystyrene target completely absorbs the laser radiation so that the skin is exposed only to the impulse transient, and not to laser radiation. No pain is produced from this procedure. The procedure can be repeated daily, or as often as required, to maintain the circulating blood levels of the agonist.

### Example 4

### Sample Collection

Selected patients were bled immediately prior to receiving the LHRH analogue to inhibit sex steroid production, and at short time intervals (typically during the first 24-72 hours) after the application of the LHRH analogue. Blood was centrifuged (750 gav) to sediment cells and the plasma collected. The plasma samples were compared by subjecting them to analysis of concentration of particular thymic marker molecules.

### Example 5

### Flow Cytometry Analysis of Whole Peripheral Blood

20 μl of the appropriate antibody cocktail was added to 200 μl whole blood and incubated in the dark, RT for 30 min. For removal of RBC, 2 ml of FACS lysis buffer (Becton-Dickinson, USA) was then added to each tube, vortexed and incubated 10 min, RT in the dark. Samples were centrifuged at 600_{gmax}, supernatant removed and cells washed twice in FACS buffer. Finally, cells were resuspended in 1% PFA for FACS analysis.

### Example 6

### Ki67 Analysis

For detection of proliferating cells, lysed samples were incubated for 20 min, RT, in the dark in 500 μl of 1× FACS permeabilising solution (Becton-Dickinson, USA). Washed samples were incubated with either anti-Ki67-PE or anti-Ki67-FITC (or the appropriate isotype controls) for 30 min at RT, in the dark. Samples were then washed and resuspended in 1% PFA for analysis.

Antibody Cocktails:

1. CD27/CD45RA/CD45RO/CD4 or CD8

2. CD62L/CD45RA/CD45RO/CD4 or CD8

3. γδTCR/αβTCR/CD28/CD4 or CD8

4. CD69/CD25/CD 152/CD3

5. CD11b/CD11c/CD56/CD3

6. CD19/CD117/CD34/CD3

7. CD3/CD4/CD8/HLA-DR

8. For Ki67:

a) CD4 or CD8/CD45RO/CD27 followed by Ki67-PE or IgG1-PE

b) αβTCR/CD8a/CD8b followed by Ki67-FITC or IgG1-PE

### Example 7

### Detection of Intracellular Cytokines

200 μl of whole blood was stimulated with soluble purified anti-CD3 (5 μg/ml) and anti-CD28 (10 μg/ml) for 6 hours at 37° C., 5% CO₂. Brefeldin A (final concentration 10 μg/ml) was added during the final 4 hours to limit cytokine secretion from the activated cells. Following stimulation, samples were incubated for 15 min, RT with 20 μl of 20 mM EDTA in PBS. Samples were then surface stained with anti-CD4-FITC and anti-CD8-CyChrome. Following lysis and permeabilisation, cells were stained with anti-IL-4-PE and anti-IFNγ-APC or the appropriate isotype controls. Unstimulated cells were used as a control for activation.

### Example 8

### Preparation of PBMC

Purified lymphocytes were used for T-cell stimulation assays and TREC analysis. 10-50 ml of peripheral blood was diluted 1:1 with RPMI-Heparin. Diluted blood was carefully layered over ficoll-hypaque at a ratio of 2:1 blood:ficoll. Tubes were centrifuged at (800_{gmax}) for 25 min at RT. Following centrifugation, the plasma layer was removed and stored at −20° C. for analysis of sex steroid levels. The buffy coat layer was removed and diluted with RPMI-Heparin. Tubes were centrifuged at 25° C. for 15 min at (600_{gmax}), followed by a second wash at 400_{gmax} for 10 min. Supernatant was removed and cell counts performed in duplicate using a haemocytometer. Cells not used for stimulation assays were resuspended in freezing media and stored at −70° C. overnight, before transferring to Liquid Nitrogen prior to TREC analysis. Plasma collected following ficoll purification was stored at −20° C. prior to analysis of sex steroid levels.

### Example 9

### T Lymphocyte Stimulation Assay

For mitogen stimulation, PBMC were plated out in 96-well round-bottom plates at a concentration of 1×10⁵/well in 100 μl of RPMI-FCS. Cells were incubated at 37° C., 5% CO₂ with PHA in doses from 1-10 μg/ml. For TCR-specific stimulation, cells were incubated for 48 hours on plates previously coated with purified anti-CD3 (1-10 μg/ml) and anti-CD28 (10 μg/ml). Following plaque formation (48-72 hours), 1 μCi of ³H-Thymidine was added to each well and plates incubated for a further 16-24 hours. Plates were harvested onto filter mats and incorporation of ³H-Thymidine was determined using liquid scintillation on a p-counter (Packard-coulter, USA).

### Example 10

### TREC Analysis

Detection of TRECs is performed by purifying new helper T cells (Th; e.g., CD4+, CD45RA+CD27+) and cytotoxic T cells (Tc; e.g., CD8+, CD45RA+CD27+) by flow cytometry and then TREC analysis using specific DNA probes and RT-PCR.

A. Cell Sorting

Frozen samples were rapidly thawed, washed in FACS buffer containing 1 mM EDTA and 1% Human Serum and centrifuged (600_{gmax}, 5 min., 4° C.). Cells were incubated with anti-CD4-FITC, anti-CD3-APC and anti-CD45RA-PE for 30 min., RT, washed and fixed by the drop-wise addition of 1 ml of 3% Formalin in PBS. Samples were incubated for a further 30 min., washed and resuspended in 500 μl FACS buffer for sorting. Four populations were obtained: CD3⁺CD4⁺CD45RA⁺; CD3⁺CD4⁺CD45RA⁻; CD3⁺CD4⁻CD45RA⁺ and CD3⁺CD4⁻CD45RA⁻.

B. DNA Isolation

Cells were sorted directly into PCR grade 0.5 ml eppendorfs, centrifuged (8 min, 2500_{gmax}) and resuspended in Proteinase K (PK) digestion buffer (2×10⁵ cells/20 μl of a 0.8 mg/mL solution). Proteinase K (PK) was added to the PCR digestion buffer just prior to use. Samples were incubated for 1 hour at 56° C. followed by 10 min at 95° C. to inactivate the proteinase. Lysed samples were stored at −70° C. prior to RT-PCR.

C. Real Time-PCR Using Molecular Beacons

This technique is described in Zhang et al., 1999. Primers for signal-joint TRECs were 5′-AAAGAGGGCAGCCCTCTCCAAGGCAAA-3′ (SEQ ID NO:1) and 5′-AGGCTGATCTTG TCTGACATTTGCTCCG-3′ (SEQ ID NO:2). Primers for coding-joint TRECs were 5′-CCTGTTTGTTAGGGCACATTAGAATCTCTCACTG-3′ (SEQ ID NO:3) and 5′-CTAATAATAAGATCCTCAAGGGTCGAGACTGTC-3′ (SEQ ID NO:4). DNA was extracted from the cells using Proteinase K digestion. PCR conditions were: 95° C. for 5 min, followed by 90° C., 60° C. and 72° C., each for 30s, for 30 or 35 cycles as indicated. Each PCR reaction contained 1U platinum Taq polymerase, 1.8 mM MgCl₂, 0.2 mM dNTPs, 12.5 μM each primer and 1.7 nmol (5 μCi) ³²P-labelled dCTP in 50 μl platinum Taq buffer.

### Example 11

### Radioimmunoassay

Detection of sex steroid levels in patient sera (frozen following Ficoll-Paque centrifugation) was performed using a ¹²⁵I-Testosterone radioimmunoassay (RIA). Prior to the assay, all reagents, samples and controls were brought to room temperature. Control tubes had either buffer alone—non-specific binding (NSB) tube or 0 ng/ml testosterone standard (B₀). Buffer alone, standards (0-10 ng/ml testosterone) or test samples were added to each tube, followed by sex binding globulin inhibitor (SBGI) to limit non-specific binding of the radio-labelled testosterone. The ¹²⁵I-testosterone was added to each tube followed by an anti-testosterone antibody (except for the NSB tubes). Tubes were then incubated at 37° C. for 2 hours. Following this, a secondary antibody was added to all tubes which were incubated for a further 60 mins following vortexing. Tubes were centrifuged (1000_{gmax}) for 15 mins, supernatant removed and the precipitate counted on a Packard Cobra auto-γ counter. Triplicate cpm results were averaged and a standard curve constructed using the formula for percent bound Testosterone (B/B₀):

$$\% B / {B}_{0} = \frac{Sample - NSB}{{B}_{0} - NSB}$$

$$\begin{matrix}Sample = average cpm of particular test sample \\ NSB = average cpm of non \text{-} specific binding tube \\ {B}_{0} = average cpm of 0 ng \text{/} ml standard ( total binding tube )\end{matrix}$$

The level of testosterone in each test sample was determined from the standard curve. The plasma was subjected to protein analysis based on 2D gel electrophoresis followed by computer based bioinformatics to determine the presence of indicators of thymic function.

### REFERENCES

Aspinall, R. 1997. Age-associated thymic atrophy in the mouse is due to a deficiency affecting rearrangement of the TCR during intrathymic T cell development. J. Immunol. 158:3037.

Berzins, S. P., Boyd, R. L. and Miller, J. F. A. P. 1998. The role of the thymus and recent thymic migrants in the maintenance of the adult peripheral lymphocyte pool. J. Exp. Med. 187:1839.

Boyd, R. L., Tucek, C. L., Godfrey, D. I., Wilson, T. J, Davidson, N. J., Bean, A. G. D., Ladyman, H. M., Ritter, M. A. and Hugo, P. 1993. The thymic microenvironment. Immunology Today 14:445.

Bruijntjes, J. P., Kuper, C. J., Robinson, J. E. and Schutirman, H. J. 1993. Epithelium-free area in the thymic cortex of rats. Dev. Immunol. 3:113.

Carayon, P., and Bord, A. 1992. Identification of DNA-replicating lymphocyte subsets using a new method to label the bromo-deoxyuridine incorporated into the DNA. J. Imm. Methods 147:225.

Douek, D. C., McFarland, R. D., Keiser, P. H., Gage, E. A., Massey, J. M., Haynes, B. F., Polis, M. A., Haase, A. T., Feinberg, M. B., Sullivan, J. L., Jamieson, B. D., Zack, J. A., Picker, L. J. and Koup, R. A. 1998. Changes in thymic function with age and during the treatment of HIV infection. Nature 396:690.

Fredrickson, G. G. and Basch, R. S. 1994. Early thymic regeneration after irradiation. Development and Comparative Immunology 18:251.

George, A. J. and Ritter, M. A. 1996. Thymic involution with ageing: obsolescence or good housekeeping? Immunol. Today 17:267.

Godfrey, D. I, Izon, D. J., Tucek, C. L., Wilson, T. J. and Boyd, R. L. 1990. The phenotypic heterogeneity of mouse thymic stromal cells. Immunol. 70:66.

Godfrey, D. I, and Zlotnik, A. 1993. Control points in early T-cell development. Immunol. Today 14:547.

Hirokawa, K. 1998. Immunity and Ageing. In Principles and Practice of Geriatric Medicine. M. Pathy, ed. John Wiley and Sons Ltd.

Hirokawa, K. and Makinodan, T. 1975. Thymic involution: the effect on T cell differentiation. J. Immunol. 114:1659.

Hirokawa, K., Utsuyama M., Kasai, M., Kurashima, C., Ishijima, S. and Zeng, Y. -X. 1994. Understanding the mechanism of the age-change of thymic function to promote T cell differentiation. Immunology Letters 40:269.

Hobbs, M. V., Weigle, W. O., Noonan, D. J., Torbett, B. E., McEvilly, R. J., Koch, R. J., Cardenas, G. J. and Ernst, D. N. 1993. Patterns of cytokine gene expression by CD4+ T cells from young and old mice. J. Immunol. 150:3602.

Homo-Delarche, R. and Dardenne, M. 1991. The neuroendocrine-immune axis. Seminars in Immunopathology.

Huiskamp, R., Davids, J. A. G. and Vos, O. 1983. Short- and long-term effects of whole body irradiation with fission neutrons or x-rays on the thymus in CBA mice. Radiation Research 95:370.

Kendall, M. D. 1988. Anatomical and physiological factors influencing the thymic microenvironment. In Thymus Update I, Vol. 1. M. D. Kendall, and M. A. Ritter, eds. Harwood Academic Publishers, p. 27.

Kurashima, C, Utsuyama, M., Kasai, M., Ishijima, S. A., Konno, A. and Hirokawa, A. 1995. The role of thymus in the aging of Th cell subpopulations and age-associated alteration of cytokine production by these cells. Int. Immunol. 7:97.

Mackall, C. L. et. al. 1995. Age, thymopoiesis and CD4+ T-lymphocyte regeneration after intensive chemotherapy. New England J. Med. 332:143.

Mackall, C. L. and Gress, R. E. 1997. Thymic aging and T-cell regeneration. Immunol. Rev. 160:91.

Penit, C. and Ezine, S. 1989. Cell proliferation and thymocyte subset reconstitution in sublethally irradiated mice: compared kinetics of endogenous and intrathymically transferred progenitors. Proc. Natl. Acad. Sci, U.S.A. 86:5547.

Penit, C., Lucas, B., Vasseur, F., Rieker, T. and Boyd, R. L. 1996. Thymic medulla epithelial cells acquire specific markers by post-mitotic maturation. Dev. Immunol. 5:25.

Plosker, G. L. and Brogden, R. N. 1994. Leuprorelin. A review of its pharmacology and therapeutic use in prostatic cancer, endometriosis and other sex hormone-related disorders. Drugs 48:930.

Randle-Barrett, E. S. and Boyd, R. L. 1994. Thymic microenvironment and lymphoid responses to sublethal irradiation. Dev. Immunol. 4:1.

Scollay, R. G., Butcher, E. C. and Weissman, I. L. 1980. Thymus cell migration. Quantitative aspects of cellular traffic from the thymus to the periphery in mice. Eur. J. Immunol. 10:210.

Shortman, K., Egerton, M., Spangrude, G. J. and Scollay, R. 1990. The generation and fate of thymocytes. Seminars in Immuno. 2:3.

Starzl, T. E., Demetris, A. J., Murase, N., Ricardi, C. and Truce, M. 1992. Cell migration, chimerism, and graft acceptance. Lancet 339:1579.

Suda, T., and Zlotnik, A. 1991. IL-7 maintains the T cell precursor potential of CD3⁻CD4⁻CD8⁻ thymocytes. J. Immunol. 146:3068.

Timm, J. A. and Thoman, M. L. 1999. Maturation of CD4+ lymphocytes in the aged microenviroment results in a memory-enriched population. J. Immunol. 162:711.

Thomson, A. W. and Lu, L. 1999. Are dendritic cells the key to liver transplant? Immunology Today 20:20.

Tosi, R., Kraft, R., Luzi, P., Cintorino, M., Fankhause, G., Hess, M. W. and Cottier, H. 1982. Involution pattern of the human thymus. 1. Size of the cortical area as a function of age. Clin. Exp. Immunol. 47:497.

van Ewijk, W., Rouse, R. V. and Weissman, I. L. 1980. Distribution of H-2 microenvironments in the mouse thymus. J. Histochem. Cytochem. 28:1089.

von Freeden-Jeffry, U., Vieira, P., Lucian, L. A., McNeil, T., Burdach, E. G. and Murray, R. 1995. Lymphopenia in interleukin (IL)-7 gene-deleted mice identifies IL-7 as a nonredundant cytokine. J. Exp. Med. 181:1519.

Wiles, M. V., Ruiz, P. and Imhof, B. A. 1992. Interleukin-7 expression during mouse thymus development. Eur. J. Immunol. 22:1037.

Zlotnik, A. and Moore, T. A. 1995. Cytokine production and requirements during T-cell development. Curr. Opin. Immunol. 7:206.

1. A method for determining susceptibility of a thymus in a patient to activation through disruption of sex steroid signaling to the thymus.

2. The method of claim 1 wherein the method of disrupting the sex steroid signaling to the thymus is through surgical castration to remove the patient's gonads.

3. The method of claim 1 wherein the method of disrupting the sex steroid signaling to the thymus is through administration of one or more pharmaceuticals.

4. The method of claim 3 wherein the pharmaceuticals are selected from the group consisting of LHRH analogs, anti-LHRH receptor antibodies, anti-LHRH vaccines and combinations thereof.

5. The method of claim 4 wherein the LHRH analogs are selected from LHRH agonists and LHRH antagonists.

6. The method of claim 5 wherein the LHRH analogs are selected from the group consisting of Eulexin, Goserelin, Leuprolide, Dioxalan derivatives, Triptorelin, Meterelin, Buserelin, Histrelin, Nafarelin, Lutrelin, Leuprorelin, Deslorelin, Abarelix, Cetrorelix, Zoladex and Leupron.

7. The method of claim 3 wherein the patient's thymus has been at least in part deactivated.

8. The method of claim 3 wherein the patient is post-pubertal.

9. The method of claim 5 wherein the LHRH antagonists are quick-acting LHRH antagonists.

10. The method of claim 9 wherein the LHRH antagonists are selected from the group consisting of Abarelix and Cetrorelix.

11. The method of claim 4 wherein reduction is induced by administration of one or more LHRH agonists and one or more LHRH antagonists.

12. The method of claim 1 comprising the step of monitoring the concentration of one or more thymopoietic cytokines in the patient's blood and/or plasma.

13. The method of claim 12 wherein the cytokine is Interleukin-7.

14. The method of claim 12 comprising the steps of
a. obtaining a blood sample from a patient prior to the disruption;
b. obtaining at least one blood sample from a patient subsequent to the disruption;
c. measuring the amount of thymopoietic cytokine(s) present in each sample; and
d. comparing the amounts of thymopoietic cytokine(s) in the samples to each other,
such that an early increase in thymopoietic cytokine(s) in the patient after disruption indicates activation of the patient's thymus.

15. The method of claim 14 wherein the increase occurs within about one week of disruption.

16. The method of claim 14 wherein the increase occurs within about 4 to 5 days of disruption.

17. The method of claim 14 wherein the increase occurs within about 2 to 3 days of disruption.

18. The method of claim 14 wherein the increase occurs within about 24 hours of disruption.

19. The method of claim 1 comprising the step of monitoring the concentration of one or more thymopoietic hormones in the patient's blood and/or plasma.

20. The method of claim 19 wherein the hormone is selected from the group consisting of thymosin, thymulin and FTS.

21. The method of claim 19 comprising the steps of
a. obtaining a blood sample from a patient prior to the disruption;
b. obtaining at least one blood sample from a patient subsequent to the disruption;
c. measuring the amount of thymopoietic hormone(s) present in each sample; and
d. comparing the amounts of thymopoietic hormone(s) in the samples to each other,
such that an increase in thymopoietic hormone(s) in the patient after disruption indicates activation of the patient's thymus.

22. The method of claim 21 wherein the increase occurs within about one week of disruption.

23. The method of claim 21 wherein the increase occurs within about 4 to 5 days of disruption.

24. The method of claim 21 wherein the increase occurs within about 2 to 3 days of disruption.

25. The method of claim 21 wherein the increase occurs within about 24 hours of disruption.

26. A method of identifying thymic factors comprising the steps of
a) obtaining a blood sample from a patient;
b) disrupting sex steroid mediated signaling to the patient's thymus;
c) obtaining at least one blood sample from the patient after disruption;
d) performing protein analysis on each sample;
e) identifying new proteins that are found in the samples taken after disruption and in lesser concentrations or not at all in samples taken before disruption.

27. The method of claim 26 wherein the blood samples are treated to separate out the plasma and the analyses are performed on the plasma samples.

28. The method of claim 27 wherein the plasma samples are subjected to two dimensional gel electrophoresis.

29. The method of claim 1 comprising the step of monitoring the production of new T cells in the patient's blood.

30. The method of claim 29 wherein the production of new T cells is monitored by detecting the presence in these cells of TRECs.

31. The method of claim 30 comprising the steps of
a) sampling the patient's blood before and after inhibition;
b) sorting the cells in samples to obtain an enhanced population of T cells;
c) isolating the DNA of the cells in the samples; and
d) performing PCR on the isolated DNA using primers specific for TRECs.

32. The method of claim 31 wherein the PCR primers are selected from the group consisting of DNA SEQ ID NO:1, DNA SEQ ID NO:2, DNA SEQ ID NO:3 and DNA SEQ ID NO:4.

33. The method of claim 31 wherein an increase in TRECs after inhibition indicates thymic activation.

34. The method of claim 33 wherein the increase occurs within about one week of disruption.

35. The method of claim 33 wherein the increase occurs within about 4 to 5 days of disruption.

36. The method of claim 33 wherein the increase occurs within about 2 to 3 days of disruption.

37. The method of claim 33 wherein the increase occurs within about 24 hours of disruption.
